(19) United States
(12) Patent Application Publication
(10) Pub. No.: US 2003/0024997 A1
(43) Pub. Date: Feb. 6, 2003

(54) Air freshening compositions, articles comprising same and methods

(75) Inventors: Robert Gary Welch, Mason, OH (US); Jiten Odhavji Dihora, Hamilton, OH (US); Errol Hoffman Wahl, Cincinnati, OH (US); Lynn Ellen Downey, Milford, OH (US); Steven Matthew Gabriel, Cincinnati, OH (US); Brent Michael Heist, Cincinnati, OH (US); Toan Trinh, Maineville, OH (US); Zaiyou Liu, West Chester, OH (US); Kristin Marie Finley, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/137,529

(22) Filed: May 2, 2002

Related U.S. Application Data
(60) Provisional application No. 60/323,302, filed on Sep. 19, 2001.
(60) Provisional application No. 60/341,128, filed on Dec. 13, 2001.
(60) Provisional application No. 60/352,807, filed on Jan. 30, 2002.
(60) Provisional application No. 60/288,767, filed on May 4, 2001.

(51) Int. Cl. A61K 7/46; A61L 9/00

(52) U.S. Cl. 239/53; 422/4; 512/1; 422/305

(57) ABSTRACT

An air freshening composition including porous carrier particles having a perfume composition entrapped therein, and a second component selected from an inert filer, hygroscopic agent, binder, coating material, moisture providing agent, and mixtures thereof. The composition may further comprise various optional components such as free perfumes, colorants, disintegrants, water swelling agents, porosity modifiers and mixtures thereof. A method for processing the compositions into a solid article includes the steps of entrapping a perfume in the porous carrier particles, heating and incorporating a carrier or binder material. The compositions may be further processed by optionally incorporating a powder inert filler and forming articles from the mixture. The articles may be formed from the compositions through prilling, extrusion, or compaction amongst other techniques. The air freshening articles and compositions will provide a sustained and controlled release of the perfume compositions over a long period of time without the use of a heat to activate the release. Articles of manufacture including the air freshening articles and various packaging are also disclosed.

### CROSS REFERENCE TO RELATED APPLICATIONS

This application claims priority under 35 U.S.C. §119(e) to U.S. Provisional Application Serial No. 60/323,302, filed Sep. 19, 2001 (Attorney Docket No. 8718P), 60/341,128, filed Dec. 13, 2001 (Attorney Docket No. 8718P2), No. 60/352,807, filed Jan. 30, 2002 and No. 60/288,767, filed May 4, 2001 (Attorney Docket No. 8541 P).

### FIELD OF THE INVENTION

The present invention relates to long lasting air-freshening compositions and systems that allow for the release of desirable fragrances into an environment. The invention additionally relates to methods for the manufacture, packaging and end consumer use of such air freshening systems.

### BACKGROUND OF TH INVENTION

Air-freshening systems that allow for the release and dispersion of volatile perfume components into the air are well known. There are several different systems currently available in the market aimed at meeting this consumer need. Key categories include solid air fresheners, liquid or gel based fresheners, wick-based vapor emanation systems and scented candles. Air freshening from the liquid, gel and wick-based systems are typically accomplished through the use of electric energy to drive volatilization and release of the perfume components into the air. Solid air fresheners rely on simultaneous evaporation and diffusion of perfume and carrier material into the surrounding environment over time. Finally, air freshening with scented candles is accomplished through the incorporation of perfume oils into the candle wax. These oils are then released as the candle is burned and displayed.

A problem with air freshening systems of the prior art is that the material released at a given time changes in amount and more importantly character over the life of the dispenser. In the case of liquid dispensers, when the liquid material to be dispensed is exposed to air, the more volatile components of the liquid rapidly evaporate and become depleted so that during the early stages of operation the dispenser diffuses a material which is rich in the more volatile constituents and relatively high in vapor phase concentration. Later in the life of such dispensers, the rate of diffusion is much lower and materials that have a low evaporation rate are diffused more prominently.

This change in character over time has been overcome by using of electrical energy (“plug-ins”). Electrical energy is used to heat a filament to provide the activation energy to force the less volatile components to evaporate at the early stages of the air freshener operation. This leads to a more complete fragrance character across the life of the air freshener. While this solves the problem with character change over time, it also brings in a new limitation. The use of electrical energy in such devices limits the perfume formulation that can be used due to safety considerations. In order to meet UL (Underwriters Lab) safety requirements, these perfume formulations are limited to materials that have a flash point above a certain temperature, typically 50° F. above the maximum filament temperature. This prevents the use of more volatile perfume components that would otherwise enhance and complete the fragrance character of the perfume composition.

In a similar fashion, solid based systems rely on the on-going evaporation, diffusion and release of both perfume and carrier to the atmosphere. During initial use, intensity is quite strong and is skewed toward the lower molecular weight, more volatile components that can more readily diffuse out from the system. As the dispenser ages and the carrier continues to evaporate, the bulk of volatile perfume components have been released leaving longer chain, less volatile components that provide much less odor intensity and different overall fragrance character.

Candles suffer from similar problems as the solid based systems. Specifically, the incorporation of perfume oil in candle wax is often difficult to achieve in a quantity that ensures the release of a suitable level of fragrance into the atmosphere during candle burning. Further, more volatile perfume components may be difficult to incorporate into the wax during candle manufacturing. Furthermore, the incorporated perfume components, particularly the smaller highly volatile perfume components, tend to volatilize from the finished candle during storage. The incorporation of larger quantities of perfume and/or perfume molecules of a relatively large size tends to soften conventional candle waxes, resulting in an undesirable loss of rigidity in the candle structure.

Due to these limitations, the fragrance market has long searched for a system that would allow for a more uniform and sustained delivery of perfume over time. The preferred system would allow for high initial release of perfume to the desired area followed by a lower sustained release over time to thereby maintain the desired odor profile. More importantly, the odor “character” or relative composition of the perfume components released into the air should remain constant with time.

It is therefore an object of the present invention to provide air freshening compositions, articles and methods of making such compositions and articles, that will overcome the problems associated with the air fresheners in the prior art. It is a more specific object to provide an energy-free air freshening system that is portable, exhibits long lasting fragrance release, and provides a means to controllably deliver highly volatile fragrance components.

It is an additional object of the present invention to provide the ability to tailor the intensity and longevity of an air freshening system. The present invention also overcomes many of the conventional limitations on the amounts and types of perfumes employed in the prior art, both in terms of the materials that may be incorporated into the article and the character of the fragrance that is released over an extended duration of time. The present invention further provides methods for manufacturing an air freshening article that produces intense and long-lasting fragrances.

### SUMMARY OF THE INVENTION

The present invention provides an air freshening composition that includes porous carrier particles having a perfume composition entrapped therein, a second component for retarding the absorption and/or adsorption of water and/or for providing moisture to the porous carrier particles, said second component selected from one or more of the following: an inert filler, a hygroscopic agent, a binder, a coating material, a moisture providing agent, and optionally, a third component selected from the group consisting of free perfume, colorant, disintegrant, water swelling agent, porosity modifier and mixtures thereof.

In a process aspect of the present invention, a process for preparing a solid air freshening article comprising porous carrier particles having a perfume composition entrapped therein is provided. The process comprising the steps of entrapping a perfume composition on the porous carrier particles, heating and adding a compatible binder or coating material to the porous carrier particles to form agglomerated particles, optionally adding an inert filler to the agglomerated particles to form a powder mixture, and optionally forming articles from the powder mixture, wherein humidity conditions throughout these steps are maintained below about 50% relative humidity (RH), preferably below about 30% RH, more preferably below about 20% RH, at a temperature of 25° C.

The present invention also provides for the use of an air freshening composition to provide a controlled release of perfume from the perfume article over a long duration of time, the composition including porous carrier particles having a perfume composition entrapped therein, a second component for retarding the absorption and/or adsorption of water and/or for providing moisture to the porous carrier particles, said second component selected from one or more of the following an inert filler, a hygroscopic agent, a binder, a coating material, a moisture providing agent, and optionally, a third component selected from the group consisting of free perfume, colorant, disintegrant, water swelling agent, porosity modifier and mixtures thereof.

In another embodiment of the present invention, an article of manufacture for deodorizing or odorizing an environment is provided. The article comprises (A) a solid air freshening article that comprises porous carrier particles, a perfume composition adhering to said porous carrier particles, an optional component selected from the group consisting of fillers, binders, coating materials, hygroscopic agents and mixtures thereof, and (B) a humidity resistant package for inhibiting moisture from contacting the porous carrier particles of the air freshening article.

In still another embodiment of the present invention, an article of manufacture for deodorizing or odorizing an environment is provided. The article comprises (A) a solid air freshening article that comprises porous carrier particles, a perfume composition adhering to said porous carrier particles, an optional component selected from the group consisting of fillers, binders, coating materials, hygroscopic agents and mixtures thereof, and (B) a package for the air freshening article having an opening therein that allows fluid communication between the air freshening article and the environment.

In a further process aspect of the present invention, a method of deodorizing and/or odorizing a room is provided. The method comprises the steps of providing a composition including porous carrier particles having a perfume composition entrapped therein, a second component for retarding the absorption and/or adsorption of water by and/or for providing moisture to the porous carrier particles, said second component selected from one or more of the following an inert filler, a hygroscopic agent, a binder, a coating material, a moisture providing agent, and optionally, a third component selected from the group consisting of free perfume, colorant, disintegrant, water swelling agent, porosity modifier and mixtures thereof. The method further comprises the steps of providing a humidity resistant package to protect the composition and providing instructions in association with the package, the instructions including an instruction to remove or open the package to expose the composition to humidity and thereby activate the release of the perfume composition to the room.

These and additional objects and advantages will be more fully apparent in view of the following detailed description. All percentages, ratios and proportions herein are by weight, unless otherwise specified. All temperatures are in degrees Celsius (° C.) unless otherwise specified. All documents cited are incorporated herein by reference in their entireties. Citation of any reference is not an admission regarding any determination as to its availability as prior art to the claimed invention.

As used herein, “comprising” means that other steps and other ingredients that do not affect the end of result can be added. This term encompasses the terms “consisting of” and “consisting essentially of”.

### DETAILED DESCRIPTION OF THE PREFERRED EMBODIMENTS

The present invention is directed to compositions and articles that are capable of delivering fragrances in a controlled manner, particularly for delivering high odor intensity into an environment for a long duration of time. More specifically, the invention is directed to an air freshening powder composition, solid air freshening articles comprising the compositions, processes for making the solid air freshening articles, articles of manufacture comprising the solid air freshening articles and methods of using the compositions and articles.

In another embodiment, the present invention is directed to methods for manufacturing an air freshening article. The methods comprise loading porous carrier particles with perfume, adding a binder and/or coating material to the porous carrier particles to form agglomerates, optionally adding an inert filler material to the agglomerates and optionally forming articles from the agglomerates, preferably forming tablets from the agglomerates using a compaction process.

In still a further embodiment, the present invention is directed to an article of manufacture comprising an air freshening article and a package for protecting the air freshening article from atmospheric moisture. In an alternative embodiment of the articles of manufacture of the present invention the article comprises an air freshening article and a package for enabling fluid communication between the air freshening article and the environment to be deodorized or odorized.

A. Perfume Delivery Composition

The inventive compositions comprise porous carrier particles having a perfume composition entrapped therein, a second component for retarding the absorption or adsorption of water by the porous carrier particles or a moisture providing material for providing moisture to the porous carrier particles, said second component selected from inert fillers, hygroscopic agents, binders, coating materials, moisture providing materials and mixtures thereof. Optionally, the composition can contain a third component selected from the group consisting of free perfumes, colorants, water swelling agents, porosity modifiers and mixtures thereof. Each of these components is described in detail below.

The compositions will deliver or release a perfume compositions to the environment to which the composition is exposed at rate between about 1 mg/hour and about 100 mg/hr, preferably at least about 10 mg/hr and more preferably at least about 15 mg/hr. Moreover, this release rate should be maintained for a prolonged period of time between about 3 days and about 28 days.

1. Perfume Composition

The porous carrier particles will have entrapped or adsorbed in the pores and/or adhered to the outer surface, a perfume composition. As described herein, the perfume composition is generally referred to as an element of the porous carrier. However, it is preferred that the air freshening compositions will comprise at least about 1%, preferably at least about 10% and more preferably at least about 20% of a perfume composition.

As used herein the term “perfume” is used to indicate any odoriferous material that is entrapped or “loaded on” the porous inorganic carrier particles for subsequent release into the atmosphere. The perfume will most often be liquid at about 250 C. A wide variety of chemicals are known for perfume uses, including materials such as aldehydes, ketones, and esters. More commonly, naturally occurring plant and animal oils and exudates comprising complex mixtures of various chemical components are known for use as perfumes. The perfumes herein can be relatively simple in their compositions or can comprise highly sophisticated complex mixtures of natural and synthetic chemical components, all chosen to provide any desired odor. Typical perfumes can comprise, for example, woody/earthy bases containing exotic materials such as sandalwood, civet and patchouli oil. The perfumes can be of a light floral fragrance, e.g. rose extract, violet extract, lilac and the like. The perfumes can also be formulated to provide desirable fruity odors, e.g. lime, lemon, and orange. Further, it is anticipated that so-called “designer fragrances” that are typically applied directly to the skin may be used as desired. As such, any material that exudes a pleasant or otherwise desirable odor can be used as a perfume active in the compositions and articles of the present invention.

The perfumes employed in the compositions and articles of the present invention can comprise ingredients that can provide an aromatherapy and/or aromachology effect. Aromatherapy effects pertain, for example, to a therapeutic treatment, while aromachology effects relate, for example, to a psychological and/or mental conditioning effect, such as providing a relaxing or invigorating mood. Perfume ingredients and/or essential oils that may provide these desired effects are described, e.g., in “The Complete Book of Essential oils & Aromatherapy”, V. A. Worwood, New World Library, San Rafael, Calif., 1991, and “The Aromatherapy Book”, J. Rose, North Atlantic Books, Berkeley, Calif., 1992, said publications are incorporated herein by reference.

In one embodiment, at least about 25%, more specifically at least about 50%, even more specifically at least about 75%, by weight of the perfume is composed of fragrance material selected from the group consisting of aromatic and aliphatic esters having molecular weights from about 130 to about 250; aliphatic and aromatic alcohols having molecular weights from about 90 to about 240; aliphatic ketones having molecular weights from about 150 to about 260; aromatic ketones having molecular weights from about 150 to about 270; aromatic and aliphatic lactones having molecular weights from about 130 to about 290; aliphatic aldehydes having molecular weights from about 140 to about 200; aromatic aldehydes having molecular weights from about 90 to about 230; aliphatic and aromatic ethers having molecular weights from about 150 to about 270; and condensation products of aldehydes and amines having molecular weights from about 180 to about 320; and essentially free from nitromusks and halogenated fragrance materials.

More specifically, in a further embodiment, at least about 25%, at least about 50%, or at least about 75%, by weight of the perfume is composed of fragrance material selected from the group consisting of those set forth in the following table:

| Common Name | Chemical Type | Chemical Name | ˜M.W. |
|---|---|---|---|
| Adoxal | aliphatic aldehyde | 2,6,10-trimethyl-9-undecen-1-al | 210 |
| allyl amyl glycolate | Ester | allyl amyl glycolate | 182 |
| allyl cyclohexane | Ester | allyl-3-cyclohexyl propionate | 196 |
| propionate | | | |
| Amyl acetate | Ester | 3-methyl-1-butanol acetate | 130 |
| Amyl salicylate | Ester | amyl salicylate | 208 |
| Anisic aldehyde | aromatic aldehyde | 4-methoxy benzaldehyde | 136 |
| Aurantiol | schiff base | condensation product of methyl | 305 |
| | | anthranilate and hydroxycitronellal | |
| Bacdanol | aliphatic alcohol | 2-ethyl-4-(2,2,3-trimethyl-3- | 208 |
| | | cyclopenten-1-yl)-2-buten-1-ol | |
| benzaldehyde | aromatic aldehyde | Benzaldehyde | 106 |
| benzophenone | aromatic ketone | Benzophenone | 182 |
| benzyl acetate | Ester | benzyl acetate | 150 |
| benzyl salicylate | Ester | benzyl salicylate | 228 |
| beta damascone | aliphatic ketone | 1-(2,6,6-trimethyl-1-cyclo-hexen-1- | 192 |
| | | yl)-2-buten-1-one | |
| beta gamma hexanol | alcohol | 3-hexen-1-ol | 100 |
| Buccoxime | aliphatic ketone | 1,5-dimethyl-oxime bicyclo[3,2,1] | 167 |
| | | octan-8-one | |
| Cedrol | alcohol | octahydro-3,6,8,8-tetramethyl-1H- | 222 |
| | | 3A,7-methanoazulen-6-ol | |
| Cetalox | Ether | dodecahydro-3A,6,6,9A- | 236 |
| | | tetramethylnaphtho[2,1B]-furan | |
| cis-3-hexenyl acetate | Ester | cis-3-hexenyl acetate | 142 |
| cis-3-hexenyl salicylate | Ester | beta, gamma-hexenyl salicylate | 220 |
| Citronellol | alcohol | 3,7-dimethyl-6-octenol | 156 |
| citronellyl nitrile | nitrile | geranyl nitrile | 151 |
| Clove stem oil | natural | | |
| Coumarin | lactone | Coumarin | 146 |
| cyclohexyl salicylate | Ester | cyclohexyl salicylate | 220 |
| Cymal | aromatic aldehyde | 2-methyl-3-(para iso propyl | 190 |
| | | phenyl)propionaldehyde | |
| Decyl aldehyde | aliphatic aldehyde | decyl aldehyde | 156 |
| Delta damascone | aliphatic ketone | 1-(2,6,6-trimethyl-3-cyclo-hexen-1- | 192 |
| | | yl)-2-buten-1-one | |
| dihydromyrcenol | alcohol | 3-methylene-7-methyl octan-7-ol | 156 |
| dimethyl benzyl carbinyl | Ester | dimethyl benzyl carbinyl acetate | 192 |
| acetate | | | |
| Ethyl vanillin | aromatic aldehyde | ethyl vanillin | 166 |
| Ethyl-2-methyl butyrate | Ester | ethyl-2-methyl butyrate | 130 |
| ethylene brassylate | macrocyclic lactone | ethylene tridecan-1,13-dioate | 270 |
| Eucalyptol | aliphatic epoxide | 1,8-epoxy-para-menthane | 154 |
| Eugenol | alcohol | 4-allyl-2-methoxy phenol | 164 |
| Exaltolide | macrocyclic lactone | cyclopentadecanolide | 240 |
| flor acetate | Ester | dihydro-nor-cyclopentadienyl | 190 |
| | | acetate | |
| Florhydral | aromatic aldehyde | 3-(3-isopropylphenyl)butanal | 190 |
| Frutene | Ester | dihydro-nor-cyclopentadienyl | 206 |
| | | propionate | |
| Galaxolide | Ether | 1,3,4,6,7,8-hexahydro-4,6,6,7,8,8- | 258 |
| | | hexamethylcyclopenta-gamma-2- | |
| | | benzopyrane | |
| gamma decalactone | lactone | 4-N-hepty-4-hydroxybutanoic acid | 170 |
| | | lactone | |
| gamma dodecalactone | lactone | 4-N-octyl-4-hydroxy-butanoic acid | 198 |
| | | lactone | |
| Geraniol | alcohol | 3,7-dimethyl-2,6-octadien-1-ol | 154 |
| geranyl acetate | Ester | 3,7-dimethyl-2,6-octadien-1-yl | 196 |
| | | acetate | |
| geranyl nitrile | Ester | 3,7-diemthyl-2,6-octadienenitrile | 149 |
| Helional | aromatic aldehyde | alpha-methyl-3,4,(methylenedioxy) | 192 |
| | | hydrocinnamaldehyde | |
| Heliotropin | aromatic aldehyde | Heliotropin | 150 |
| Hexyl acetate | Ester | hexyl acetate | 144 |
| Hexyl cinnamic aldehyde | aromatic aldehyde | alpha-n-hexyl cinnamic aldehyde | 216 |
| Hexyl salicylate | Ester | hexyl salicylate | 222 |
| hydroxyambran | aliphatic alcohol | 2-cyclododecyl-propanol | 226 |
| hydroxycitronellal | aliphatic aldehyde | hydroxycitronellal | 172 |
| ionone alpha | aliphatic ketone | 4-(2,6,6-trimethyl-1-cyclohexenyl-1- | 192 |
| | | yl)-3-buten-2-one | |
| ionone beta | aliphatic ketone | 4-(2,6,6-trimethyl-1-cyclohexen-1- | 192 |
| | | yl)-3-butene-2-one | |
| ionone gamma methyl | aliphatic ketone | 4-(2,6,6-trimethyl-2-cyclohexyl-1- | 206 |
| | | yl)-3-methyl-3-buten-2-one | |
| iso E super | aliphatic ketone | 7-acetyl-1,2,3,4,5,6,7,8-octahydro- | 234 |
| | | 1,1,6,7,tetramethyl naphthalene | |
| iso eugenol | Ether | 2-methoxy-4-(1-propenyl)phenol | 164 |
| iso jasmone | aliphatic ketone | 2-methyl-3-(2-pentenyl)-2- | 166 |
| | | cyclopenten-1-one | |
| Koavone | aliphatic aldehyde | acetyl di-isoamylene | 182 |
| Lauric aldehyde | aliphatic aldehyde | lauric aldehyde | 184 |
| Lavandin | natural | | |
| Lavender | natural | | |
| Lemon CP | natural | major component | |
| | | d-limonene | |
| d-limonene/orange | ailcene | 1-methyl-4-iso-propenyl-1- | 136 |
| terpenes | | cyclohexene | |
| Linalool | alcohol | 3-hydroxy-3,7-dimethyl-1,6- | 154 |
| | | octadiene | |
| linalyl acetate | Ester | 3-hydroxy-3,7-dimethyl-1,6- | 196 |
| | | octadiene acetate | |
| lrg 201 | Ester | 2,4-dihydroxy-3,6-dimethyl benzoic | 196 |
| | | acid methyl ester | |
| Lyral | aliphatic aldehyde | 4-(4-hydroxy-4-methyl-pentyl)3- | 210 |
| | | cylcohexene-1-carboxaldehyde | |
| Majantol | aliphatic alcohol | 2,2-dimethyl-3-(3-methylphenyl)- | 178 |
| | | propanol | |
| Mayol | alcohol | 4-(1-methylethyl)cyclohexane | 156 |
| | | methanol | |
| methyl anthranilate | aromatic amine | methyl-2-aminobenzoate | 151 |
| methyl beta naphthyl | aromatic ketone | methyl beta naphthyl ketone | 170 |
| ketone | | | |
| methyl cedrylone | aliphatic ketone | methyl cedrenyl ketone | 246 |
| methyl chavicol | Ester | 1-methyloxy-4,2-propen- | 148 |
| | | 1-yl benzene | |
| methyl dihydro jasmonate | aliphatic ketone | methyl dihydro jasmonate | 226 |
| methyl nonyl | aliphatic aldehyde | methyl nonyl acetaldehyde | 184 |
| acetaldehyde | | | |
| Musk indanone | aromatic ketone | 4-acetyl-6-tert butyl-1,1-dimethyl | 244 |
| | | indane | |
| Nerol | alcohol | 2-cis-3,7-dimethyl-2,6-octadien-1-ol | 154 |
| Nonalactone | lactone | 4-hydroxynonanoic acid, lactone | 156 |
| Norlimbanol | aliphatic alcohol | 1-(2,2,6-trimethyl-cyclohexyl)-3- | 226 |
| | | hexanol | |
| orange CP | natural | major component | |
| | | d-limonene | |
| P.T. bucinal | aromatic aldehyde | 2-methyl-3(para tert butylphenyl) | 204 |
| | | propionaldehyde | |
| para hydroxy phenyl | aromatic ketone | para hydroxy phenyl butanone | 164 |
| butanone | | | |
| Patchouli | natural | | |
| phenyl acetaldehyde | aromatic aldehyde | 1-oxo-2-phenylethane | 120 |
| phenyl acetaldehyde | aromatic aldehyde | phenyl acetaldehyde dimethyl acetyl | 166 |
| dimethyl acetyl | | | |
| phenyl ethyl acetate | Ester | phenyl ethyl acetate | 164 |
| phenyl ethyl alcohol | alcohol | phenyl ethyl alcohol | 122 |
| phenyl ethyl phenyl | Ester | 2-phenylethyl phenyl acetate | 240 |
| acetate | | | |
| phenyl | alcohol | 3-methyl-5-phenylpentanol | 178 |
| hexanol/phenoxanol | | | |
| Polysantol | aliphatic alcohol | 3,3-dimethyl-5-(2,2,3-trimethyl-3- | 221 |
| | | cyclopenten- | |
| | | 1-yl)-4-penten-2-ol | |
| phenyl acetate | Ester | 2-methylbuten-2-ol-4-acetate | 128 |
| Rosaphen | aromatic alcohol | 2-methyl-5-phenyl pentanol | 178 |
| Sandalwood | natural | | |
| alpha-terpinene | aliphatic alkane | 1-methyl-4-iso- | 136 |
| | | propylcyclohexadiene-1,3 | |
| terpineol (alpha terpineol | alcohol | para-menth-1-en-8-ol, para-menth-1- | 154 |
| and beta terpineol) | | en-1-ol | |
| terpinyl acetate | Ester | para-menth-1-en-8-yl acetate | 196 |
| tetra hydro linalool | aliphatic alcohol | 3,7-dimethyl-3-octanol | 158 |
| tetrahydromyrcenol | aliphatic alcohol | 2,6-dimethyl-2-octanol | 158 |
| tonalid/musk plus | aromatic ketone | 7-acetyl-1,1,3,4,4,6-hexamethyl | 258 |
| | | tetralin | |
| undecalactone | lactone | 4-N-heptyl-4-hydroxybutanoic acid | 184 |
| | | lactone | |
| Undecavertol | alcohol | 4-methyl-3-decen-5-ol | 170 |
| undecyl aldehyde | aliphatic aldehyde | Undecanal | 170 |
| undecylenic aldehyde | aliphatic aldehyde | undecylenic aldehyde | 168 |
| Vanillin | aromatic aldehyde | 4-hydroxy-3-methoxybenzaldehyde | 152 |
| Verdox | Ester | 2-tert-butyl cyclohexyl acetate | 198 |
| Vertenex | Ester | 4-tert-butyl cyclohexyl acetate | 198 |

Perfume compositions useful in the compositions and articles of the present invention preferably comprise at least about 6 perfume ingredients, more preferably at least about 7 ingredients, still more preferably at least about 8 and even more preferably at least about 9 ingredients. It is often desirable in the air freshening industry to incorporate highly volatile perfumes. Perfume agents may therefore be further identified on the basis of their volatility. Boiling point is used herein as a measure of volatility.

Conventional air freshening devices are not capable of delivering fully formulated fragrance character from an air freshener over a long period of time. Gel air fresheners, potpourri products, and scented candles generally provide distillative release of fragrances wherein the more volatile notes are initially released into the environment, followed by molecules of lower volatility. Because of this distillative release, the consumer generally does not detect the full character of the perfume or a uniform scent over the life of the air freshener. Electric air freshening devices overcome these obstacles at the expense of portability; in addition, safety regulations seriously restrict the fragrance formulation flexibility in such devices.

The present invention overcomes these obstacles by providing compositions and articles in which perfume components of low boiling point or flash point can be incorporated to safely deliver the fully formulated fragrance characters to the environment. More specifically, the present invention allows the incorporation of the typically avoided highly volatile perfumes. Thus, in another embodiment of the present invention, the perfume composition herein typically comprises at least about 25%, preferably at least about 40%, more preferably at least about 60%, and even more preferably at least about 75%, by weight of the perfume composition, of diffusive perfume ingredients. A diffusive perfume ingredient is characterized by its boiling point (B.P.) of about 250° C. or lower, determined at the normal, standard pressure of about 760 mm Hg. When diffusive perfume ingredients having a B.P. of about 250° C. or lower are used in a perfume composition, the ingredients are very effusive when used without means for controlling the rate of release. The perfume carrier of the present invention is controls the rate of release of such diffusive perfume ingredients.

The boiling points of many perfume ingredients are given in, e.g., “Perfume and Flavor Chemicals (Aroma Chemicals),” Steffen Arctander, published by the author, 1969, incorporated herein by reference. Other boiling point values can be obtained from different chemistry handbooks and data bases, such as the Beilstein Handbook, Lange's Handbook of Chemistry, and the CRC Handbook of Chemistry and Physics. When a boiling point is given only at a different pressure, usually lower pressure than the normal pressure of 760 mm Hg, the boiling point at normal pressure can be approximately estimated by using boiling point-pressure monographs, such as those given in “The Chemist's Companion,” A. J. Gordon and R. A. Ford, John Wiley & Sons Publishers, 1972, pp. 30-36. The boiling point values can also be estimated via a computer program that is described in “Development of a Quantitative Structure·Property Relationship Model for Estimating Normal Boiling Points of Small Multifunctional Organic Molecules”, David T. Stanton, Journal of Chemical Information and Computer Sciences, Vol. 40, No. 1, 2000, pp. 81-90, which incorporated herein by reference.

Non-limiting examples of diffusive perfume ingredients that are useful in the composition of the present invention are allyl caproate, allyl heptoate, amyl acetate, amyl propionate, anethol, anisic aldehyde, anisole, benzaldehyde, benzyl acetate, benzyl acetone, benzyl alcohol, benzyl butyrate, benzyl formate, benzyl iso valerate, benzyl propionate, camphene, camphor gum, carvacrol, laevo-carveol, d-carvone, laevo-carvone, cinnamyl formate, citral (neral), citronellol, citronellyl acetate, citronellyl isobutyrate, citronellyl nitrile, citronellyl propionate, para-cresol, para-cresyl methyl ether, cyclohexyl ethyl acetate, cuminic alcohol, cuminic aldehyde, cyclal C (3,5-dimethyl-3-cyclohexene-1-carboxaldehyde), para-cymene, decyl aldehyde, dihydro myrcenol, dihydromyrcenyl acetate, dimethyl benzyl carbinol, dimethyl benzyl carbinyl acetate, dimethyl octanol, diphenyl oxide, dodecalactone, ethyl acetate, ethyl aceto acetate, ethyl amyl ketone, ethyl benzoate, ethyl butyrate, ethyl hexyl ketone, ethyl methyl phenyl glycidate, ethyl phenyl acetate, eucalyptol, eugenol, fenchyl acetate, fenchyl alcohol, flor acetate (tricyclo decenyl acetate), frutene (tricyclo decenyl propionate), geraniol, geranyl acetate, geranyl formate, geranyl isobutyrate, geranyl nitrile, hexenol, beta gamma hexenol, hexenyl acetate, cis-3-hexenyl acetate, hexenyl isobutyrate, cis-3-hexenyl tiglate, hexyl acetate, hexyl formate, hexyl neopentanoate, hexyl tiglate, hydratropic alcohol, hydroxycitronellal, indole, alpha-ionone, beta-ionone, gamma-ionone, alpha-irone, isoamyl alcohol, isobomyl acetate, isobutyl benzoate, isomenthone, isononyl acetate, isononyl alcohol, isobutyl quinoline, isomenthol, para-isopropyl phenylacetaldehyde, isopulegol, isopulegyl acetate, isoquinoline, cisjasmone, lauric aldehyde (dodecanal), ligustral (2,4-dimethyl-3-cyclohexene-1-carboxaldehyde), d-limonene, linalool, linalool oxide, linalyl acetate, linalyl formate, menthone, menthyl acetate, methyl acetophenone, para-methyl acetophenone, methyl amyl ketone, methyl anthranilate, methyl benzoate, methyl benzyl acetate, methyl chavicol, methyl eugenol, methyl heptenone, methyl heptine carbonate, methyl heptyl ketone, methyl hexyl ketone, gamma methyl ionone, gamma-n-methyl ionone, alpha-iso gamma-methyl ionone, methyl nonyl acetaldehyde, methyl octyl acetaldehyde, methyl phenyl carbinyl acetate, methyl salicylate, myrcene, neral, nerol, neryl acetate, gamma-nonalactone, nonyl acetate, nonyl aldehyde, allo-ocimene, octalactone, octyl alcohol (octanol-2), octyl aldehyde, orange terpenes (d-limonene), phenoxy ethanol, phenyl acetaldehyde, phenyl ethyl acetate, phenyl ethyl alcohol, phenyl ethyl dimethyl carbinol, alpha-pinene, beta-pinene, prenyl acetate, propyl butyrate, pulegone, rose oxide, safrole, alpha-terpinene, gamma-terpinene, 4-terpinenol, alpha-terpineol, terpinolene, terpinyl acetate, tetrahydro linalool, tetrahydro myrcenol, tonalid (6-acetyl-1,1,3,4,4,6-hexamethyl tetrahydronaphthalene), undecenal, veratrol (ortho-dimethoxybenzene), verdox (2-tert-butylcyclohexyl acetate), vertenex (4-tert-butyl cyclohexyl acetate), viridine (phenylacetaldehyde dimethylacetal), and mixtures thereof.

In the perfume art, some auxiliary materials having no odor, or a low odor, are used, e.g., as solvents, diluents, extenders or fixatives. Non-limiting examples of these materials are ethyl alcohol, carbitol, diethylene glycol, dipropylene glycol, diethyl phthalate, triethyl citrate, isopropyl myristate, and benzyl benzoate. These materials are used, e.g., for solubilizing or diluting some solid or viscous perfume ingredients, e.g., to improve handling and/or formulating. These auxiliary materials can be useful in the diffusive perfume compositions, but are not counted in the calculation of the limits for the definition/formulation of the diffusive perfume compositions of the present invention. Perfume fixatives are traditionally used in an attempt to slow the evaporation of more volatile components of the perfume. In the present invention, the release of diffusive perfume ingredients is mediated by the porous perfume carrier. Therefore, perfume fixatives are normally not needed. However, they can be useful in the optional free perfume composition.

Non-diffusive perfume ingredients are those having a B.P. of more than about 250° C. In some compositions, some non-diffusive perfume ingredients can be used, e.g., to improve perfume odor character.

The perfumes useful in the present invention compositions are preferably substantially free of halogenated materials and nitromusks.

Further, not to be limited by theory, hydrocarbons nonspecifically adsorb within the cavities of zeolite X, although there is some tendency for them to associate with the Na⁺ ions. Molecules with aromatic rings (net negatively charged carbon atoms) interact with the Na⁺ ions electrostatically, and result in moderate adsorption. Polar adsorbates, such as acetone, have a very strong preference to associate with the charge density centers in the zeolite cavities (occupied by Na⁺ ions) in a manner similar to that observed for water. Hence, perfume raw materials that are polar in character, e.g., having a non-zero dipole moment or dipole-dipole interaction, preferably interact with the Na⁺ ions, and are strongly adsorbed into the zeolite cavity. This strong adsorption translates to the ability to control the release of these components from the zeolite. Therefore, it is preferred that the perfume composition comprise at least about 25%, preferably at least about 50% and more preferably at least about 75% by weight of perfume components having a dipole-dipole interaction or a non-zero dipole moment.

The perfume composition can additionally comprise perfume ingredients with low odor detection threshold. The odor detection threshold of an odorous material is the lowest vapor concentration of that material which can be olfactorily detected. The odor detection threshold and some odor detection threshold values are discussed in, e.g., “Standardized Human Olfactory Thresholds”, M. Devos et al, IRL Press at Oxford University Press, 1990, and “Compilation of Odor and Taste Threshold Values Data”, F. A. Fazzalari, editor, ASTM Data Series DS 48A, American Society for Testing and Materials, 1978, both of said publications being incorporated by reference. The use of small amounts of perfume ingredients that have low odor detection threshold values can improve perfume odor character, and are especially useful in the compositions of the present invention. These materials can be present at low levels in the perfume compositions of the present invention, typically less than about 20% by weight of the total perfume compositions of the present invention.

Nonlimiting examples of perfume ingredients that have a significantly low detection threshold, useful in the composition of the present invention, are allyl amyl glycolate, ambrox (1,5,5,9-tetramethyl-1,3-oxatricyclotridecane), anethole, bacdanol (2-ethyl-4-(2,2,3-trimethyl-3-cyclopenten-1-yl)-2-buten-1-ol), benzyl acetone, benzyl salicylate, butyl anthranilate, calone, cetalox (2-ethyl-4-(2,2,3-trimethyl-3-cyclopenten-1-yl)-2-buten-1-ol), cinnamic alcohol, coumarin, cyclogalbanate, Cyclal C (3,5-dimethyl-3-cyclohexene-1-carboxaldehyde), cymal (2-methyl-3-(para isopropylphenyl)propionaldehyde), damascenone (1-(2,6,6-trimethyl-1,3-cyclohexadien-1-yl)-2-buten-1-one), alpha-damascone, 4-decenal, dihydro isojasmonate, gamma-dodecalactone, ebanol, ethyl anthranilate, ethyl-2-methyl butyrate, ethyl methylphenyl glycidate, ethyl vanillin, eugenol, flor acetate (dihydro-nor-cyclopentadienyl acetate), florhydral (3-(3-isopropylphenyl)butanol), fructone (ethyl-2-methyl-1,3-dioxolane-2-acetate), frutene (dihydro-nor-cyclopentadienyl propionate), heliotropin, herbavert (3,3,5-trimethylcyclohexyl-ethyl ether), cis-3-hexenyl salicylate, indole, alpha-ionone, beta-ionone, iso cyclo citral, isoeugenol, alpha-isomethylionone, keone, lilial (para-tertiary butyl alpha-methyl hydrocinnamic aldehyde), linalool, lyral (4-(4-hydroxy-4-methyl-pentyl)3-cylcohexene-1-carboxaldehyde), methyl heptine carbonate, methyl anthranilate, methyl dihydrojasmonate, methyl isobutenyl tetrahydropyran, methyl beta naphthyl ketone, methyl nonyl ketone, beta naphthol methyl ether, nerol, para-anisic aldehyde, para hydroxy phenyl butanone, phenyl acetaldehyde, gamma-undecalactone, undecylenic aldehyde, vanillin, and mixtures thereof. Some of the low odor detection perfume ingredients are also diffusive perfume ingredients.

Following are nonlimiting examples of diffusive perfume compositions of the present invention:

| Perfume Ingredients | Wt. % | |
|---|---|---|
| PERFUME A - Citrus Floral Type | | |
| Amyl salicylate | 1 | |
| Anisic aldehyde | 1 | |
| Citral | 4 | |
| Citronellol | 5 | |
| Citronellyl nitrile | 3 | |
| para Cymene | 2 | |
| Decyl aldehyde | 1 | |
| Dihydro myrcenol | 15 | |
| Geranyl nitrile | 3 | |
| beta gamma Hexenol | 0.3 | |
| cis-3-Hexenyl acetate | 0.2 | |
| Hexyl cinnamic aldehyde | 5 | |
| Hexyl salicylate | 3 | |
| alpha-Ionone | 2 | |
| cis-Jasmone | 1 | |
| Linalool | 8 | |
| Linalyl acetate | 5 | |
| gamma-Methyl ionone | 3 | |
| Myrcene | 1.5 | |
| Nerol | 3 | |
| Orange terpenes | 15 | |
| P.T. Bucinal | 5 | |
| Patchouli | 1 | |
| Phenyl hexanol | 5 | |
| beta-Pinene | 3 | |
| alpha-Terpineol | 4 | |
| Total | 100 | |
| PERFUME B - Rose Floral Type | | |
| Aurantiol | 3 | |
| Benzophenone | 3 | |
| Citronellol | 15 | |
| Citronellyl nitrile | 3 | |
| Decyl aldehyde | 1 | |
| Dihydro myrcenol | 5 | |
| Dimethyl octanol | 5 | |
| Diphenyl oxide | 1 | |
| Geraniol | 7 | |
| Geranyl acetate | 3 | |
| Geranyl formate | 3 | |
| Hexyl cinnamic aldehyde | 10 | |
| alpha-Ionone | 3 | |
| Isobornyl acetate | 4 | |
| gamma-Methyl ionone | 4 | |
| P.T. Bucinal | 10 | |
| Phenyl ethyl alcohol | 15 | |
| Terpineol | 5 | |
| Total | 100 | |
| PERFUME C - Natural Lime Type | | |
| Bisabolene | 3 | |
| Camphene | 1 | |
| Caryophyllene | 1 | |
| para-Cymene | 1 | |
| Eucalyptol | 1.5 | |
| Fenchyl alcohol | 1 | |
| Geranyl acetate | 2 | |
| d-Limonene | 49 | |
| Linalool | 3 | |
| Myrcene | 2 | |
| alpha-Pinene | 1.5 | |
| beta-Pinene | 2 | |
| Terpinene-4-ol | 2 | |
| Terpineol | 10 | |
| Terpinolene | 20 | |
| Total | 100 | |
| PERFUME D - Natural Lemon Type | | |
| Citral | 4 | |
| Frutene | 15 | |
| d-Limonene | 50 | |
| Linalyl Acetate | 6 | |
| Methyl Dihydrojasmonate | 18 | |
| alpha-Pinene | 4 | |
| beta-Pinene | 3 | |
| Total | 100 | |
| PERFUME E - Fruity Lemon Type | | |
| Camphor gum | 0.5 | |
| para-Cymene | 0.5 | |
| Dihydro myrcenol | 1 | |
| Dihydro terpineol | 2.5 | |
| Dimethyl benzyl carbinol | 1 | |
| Dimetol | 1.5 | |
| Eucalyptol | 1 | |
| Fenchyl alcohol | 1.5 | |
| Isononyl alcohol | 0.5 | |
| Tetrahydro linalool | 45 | |
| Tetrahydro myrcenol | 44 | |
| Verdox | 1 | |
| Total | 100 | |
| PERFUME F - Citrus Lime Type | | |
| Benzyl Propionate | 2 | |
| Citral | 3 | |
| Citronellyl nitrile | 2 | |
| Decyl aldehyde | 0.5 | |
| Dihydro myrcinol | 10 | |
| Eucalyptol | 2 | |
| Fenchyl alcohol | 0.5 | |
| Flor acetate | 7 | |
| Frutene | 5 | |
| Geranyl nitrile | 3 | |
| beta gamma Hexenol | 0.5 | |
| Linalool | 7 | |
| Linalyl acetate | 5 | |
| Methyl dihydro jasmonate | 5 | |
| Octyl aldehyde | 0.5 | |
| Orange terpenes | 30 | |
| para-Cymene | 1.5 | |
| Phenyl hexanol | 5 | |
| alpha-Pinene | 2.5 | |
| alpha-Terpineol | 2 | |
| Terpinyl acetate | 2 | |
| Tetrahydro linalool | 3 | |
| Verdox | 1 | |
| Total | 100 | |
| PERFUMES G and H - Freshening Type | | |
| | | H |
| | G | Wt. % |
| Amyl salicylate | 8 | — |
| Benzyl acetate | 8 | 8 |
| Benzyl Salicylate | — | 2 |
| Citronellol | 7 | 25 |
| Dihydromyrcenol | 2 | — |
| Eugenol | 4 | — |
| Flor acetate | 8 | — |
| Galaxolide | 1 | — |
| Geraniol | 5 | — |
| Hexyl cinnamic aldehyde | 2 | — |
| Hydroxycitronellal | 3 | — |
| Lilial | 2 | — |
| Linalool | 6 | 9 |
| Linalyl acetate | 5 | — |
| Lyral | 3 | — |
| Methyl dihydrojasmonate | 3 | — |
| Nerol | 2 | — |
| Orange terpenes | 7 | 10 |
| Phenoxy ethyl propionate | — | 3 |
| Phenylethyl acetate | 5 | 15 |
| Phenylethyl alcohol | 7 | 15 |
| alpha-Terpineol | 5 | 13 |
| aipha-Terpinene | 5 | — |
| Tetrahydromyrcenol | 2 | — |
| Total | 100 | 100 |

Therefore, the perfume compositions will preferably comprise at least two High Efficiency Air Bloom (HEAB) perfume ingredients, each having either (a) a standard B.P. of about 250° C. or lower at 760 mm Hg, or (b) an Odor Detection Threshold (ODT) of less than or equal to 50 ppb. Perfume compositions comprising HEAB ingredients are very effusive and very noticeable. Of the perfume ingredients in a given perfume oil, at least about 40%, preferably at least about 50% and most preferably at least about 70% are HEAB perfume ingredients.

Non-limiting examples of (HEAB) perfume ingredients include 4-(2,2,6-Trimethylcyclohex-1-enyl)-2-en-4-one, 2,4-Decadienoic acid, ethyl ester (E,Z)-6-(and -8) isopropylquinoline, Acetaldehyde phenylethyl propyl acetal, Acetic acid, (2-methylbutoxy)-, 2-propenyl ester, Acetic acid, (3-methylbutoxy)-, 2-propenyl ester, 2,6,10-Trimethyl-9-undecenal, Glycolic acid, 2-pentyloxy-, allyl ester, Hexanoic acid, 2-propenyl ester, 1-Octen-3-ol, trans-Anethole, iso buthyl (z)-2-methyl-2-butenoate, Anisaldehyde diethyl acetal, Benzenepropanal, 4-(1,1-dimethylethyl)-2,6-Nonadien-1-ol, 3-methyl-5-propyl-cyclohexen-1-onre, Butanoic acid, 2-methyl-, 3-hexenyl ester, (Z)-Acetaldehyde, [(3,7-dimethyl-6-ctenyl)oxy]-Lauronitrile, 2,4-dimethyl-3-cyclohexene-1-carbaldehyde, 2-Buten-1-one, 1-(2,6,6-trimethyl-1,3-cyclohexadien-1-yl)-2-Buten-1-one, 1-2,6,6-trimethyl-2-cyclohexen-1-yl)-, (E)-gamma-Decalactone, trans-4-decenal, decanal, 2-Pentylcyclopentanone, 1-(2,6,6 Trimethyl 3 Cyclohexen-1-yl)-2 Buten-1-one), 2,6-dimethylheptan-2-ol, Benzene, 1,1′-oxybis-4-Penten-1-one, 1-(5,5-dimethyl-1-cyclohexen-1-yl)-Butanoic acid, 2-methyl-, ethyl ester, Ethyl anthranilate, 2-Oxabicyclo[2.2.2]octane, 1,3,3-trimethyl-Eugenol, 3-(3-isopropylphenyl)butanal, methyl 2-octynoate, 4-(2,6,6-trimethyl-1-cyclohexen-1-yl-3-buten-2-one, Pyrazine, 2-methoxy-3-(2-methylpropyl)-Quiniline, 6-secondary buty, Isoeugenol, 2H-Pyran-2-one, tetrahydro-6-(3-pentenyl)-Cis-3-Hexenyl Methyl Carbonate, Linalool, 1,6,10-Dodecatriene, 7,11-dimethyl-3-methylene-, (E)-2,6-dimethyl-5-heptenal, 4,7 Methanoindan 1-carboxaldehyde, hexahydro 2-methylundecanal, methyl 2-nonynonate, 1,1-dimethoxy-2,2,5-trimethyl-4-hexene, Benzoic acid, 2-hydroxy-, methyl ester, 4-Penten-1-one, 1-(5,5-dimethyl-1-cyclohexen-1-yl), 2H-Pyran, 3,6-dihydro-4 methyl-2-(2-methyl-1-propenyl)-2,6-Octadienenitrile, 3,7-dimethyl-, (Z)-2,6-nonadienal, 6-Nonenal, (Z)-nonanal, octanal, 2-Nonenenitrile, Acetic acid, 4-methylphenyl ester, Gamma Undecalactone, 2-norpinene-2-propionaldehyde 6,6 dimethyl, 4-nonanolide, 9-decen-1-ol, 2H-Pyran, tetrahydro-4-methyl-2-(2-methyl-1-propenyl)-5-methyl-3-heptanone oxime, Octanal, 3,7-dimethyl-4-methyl-3-decen-5-ol, 10-Undecen-1-al, Pyridine, 2-(1-ethylpropyl)-Spiro[furan-2 (3H), 5′-[4,7]methano[5H]indene], decahydro.

The following are additional non-limiting examples of suitable perfume compositions for use in the compositions and articles of the present invention:

### EXAMPLE I

| HEAB Perfume Ingredient | Conc. | | Boiling Point |
|---|---|---|---|
| Trade Name | Wt. % | ODT | ° C. |
| Linalool | 40 | <50 PPB | 197 |
| Flor acetate | 20 | <50 PPB | 245 |
| Methyl dihydro Jasmonate | 10 | <50 PPB | |
| Ethyl-2 methyl butyrate | 10 | <50 PPB | 129 |
| Rose Oxide | 10 | <50 PPB | 201 |
| Delta Damascone | 5 | <50 PPB | 260 |
| Cyclal C | 5 | <50 PPB | 199 |
| Total | 100 | | |

### EXAMPLE J

| HIA Perfume Ingredient | Conc. | | Boiling Point |
|---|---|---|---|
| Trade Name | Wt. % | ODT | ° C. |
| Cyclal C | 10 | <50 PPB | 199 |
| Damascone Alpha | 5 | <50 PPB | 255 |
| Rose Oxide | 10 | <50 PPB | 201 |
| Benzyl Acetone | 30 | <50 PPB | 234 |
| Nerol | 30 | <50 PPB | ? |
| Methyl Octine Carbonate | 5 | <50 PPB | 219 |
| Flor acetate | 10 | <50 PPB | 245 |
| Total | 100 | | |

### EXAMPLE K

| HIA Perfume Ingredient | Conc. | | Boiling Point |
|---|---|---|---|
| Trade Name | Wt. % | ODT | ° C. |
| Damascone Alpha | 5 | <50 PPB | 250 |
| Cyclal C | 5 | <50 PPB | 199 |
| Rose Oxide | 10 | <50 PPB | 201 |
| Ionone Beta | 25 | <50 PPB | 265 |
| Flor acetate | 15 | <50 PPB | 245 |
| Anisic Aldehyde | 10 | <50 PPB | 249 |
| Ethyl-2-methyl Butyrate | 5 | <50 PPB | 129 |
| Benzyl Acetone | 25 | <50 PPB | 234 |
| Total | 100 | | |

In addition, it has now discovered that surprisingly, many common perfume ingredients are not compatible with porous carrier materials, such as clays and zeolites, particularly dehydrated/activated zeolites. It is found that some perfume ingredients are degraded upon incorporation into a porous mineral carrier material, forming materials that are undesirable and/or not intended in the original perfume compositions. Furthermore, some of these ingredients can cause discoloration in some consumable compositions.

An “unstable” perfume ingredient can be identified by loading a liquid perfume composition comprising at least 6 perfume ingredients including the perfume ingredient being studied into a sample of activated/dehydrated zeolite 13×, according to the procedure given hereinbelow, and stored under anhydrous condition for about 24 hours. The perfume ingredients are then extracted with acetone to be recovered as free perfume and analyzed by gas chromatography to determine its stability. A perfume ingredient is characterized as an “unstable perfume ingredient” if at least about 50% of that ingredient, preferably at least 65%, more preferably at least about 80%, and even more preferably at least about 95% of that ingredient is decomposed into other by-products, and not recovered from the extraction.

Non-limiting examples of the unstable perfume ingredients that are not suitable for use in the present invention preferably include ingredients selected from the group consisting of allylic alcohol ester, secondary alcohol ester, tertiary alcohol ester, allylic ketone, condensation product of amines and aldehydes, and mixtures thereof, and more preferably include ingredients selected from the group consisting of allylic alcohol ester, secondary alcohol ester, tertiary alcohol ester, allylic ketone, acetal, ketal, condensation product of amines and aldehydes, and mixtures thereof.

“Allylic alcohol” refers to an alcohol molecule wherein the carbon atom carrying the alcoholic hydroxyl group is covalently bonded to a carbon-carbon double bond in the alpha and beta positions, namely, having the general structure C(OH)—C═C. Non-limiting examples of allylic alcohol ester perfume ingredients include allyl amyl glycolate, allyl anthranilate, allyl benzoate, allyl butyrate, allyl caprate, allyl caproate, allyl cinnamate, allyl cyclohexane acetate, allyl cyclohexane butyrate, allyl cyclohexane propionate, allyl heptoate, allyl nonanoate, allyl salicylate, amyl cinnamyl acetate, amyl cinnamyl formate, cinnamyl formate, cinnamyl acetate, cyclogalbanate, geranyl acetate, geranyl acetoacetate, geranyl benzoate, geranyl cinnamate, methallyl butyrate, methallyl caproate, neryl acetate, neryl butyrate, amyl cinnamyl formate, alpha-methyl cinnamyl acetate, methyl geranyl tiglate, mertenyl acetate, farnesyl acetate, fenchyl acetate, geranyl anthranilate, geranyl butyrate, geranyl iso-butyrate, geranyl caproate, geranyl caprylate, geranyl ethyl carbonate, geranyl formate, geranyl furoate, geranyl heptoate, geranyl methoxy acetate, geranyl pelargonate, geranyl phenylacetate, geranyl phthalate, geranyl propionate, geranyl iso-propoxyacetate, geranyl valerate, geranyl iso-valerate, trans-2-hexenyl acetate, trans-2-hexenyl butyrate, trans-2-hexenyl caproate, trans-2-hexenyl phenylacetate, trans-2-hexenyl propionate, trans-2-hexenyl tiglate, trans-2-hexenyl valerate, beta-pentenyl acetate, alpha-phenyl allyl acetate, prenyl acetate, trichloromethylphenylcarbinyl acetate, and mixtures thereof.

“Secondary alcohol” refers to an alcohol moleculewherein the carbon atom carrying the alcoholic hydroxyl group is covalently bonded to a hydrogen atom and two carbon atoms, namely, having the general structure C—CH(OH)—C. Non-limiting examples of secondary alcohol ester perfume ingredients include secondary-n-amyl acetate, ortho-tertiary-amyl cyclohexyl acetate, isoamyl benzyl acetate, secondary-n-amyl butyrate, amyl vinyl carbinyl acetate, amyl vinyl carbinyl propionate, cyclohexyl salicylate, dihydro-nor-cyclopentadienyl acetate, dihydro-nor-cyclopentadienyl propionate, isobornyl acetate, isobornyl salicylate, isobornyl valerate, flor acetate, frutene, 2-methylbuten-2-ol-4-acetate, methyl phenyl carbinyl acetate, 2-methyl-3-phenyl propan-2-yl acetate, prenyl acetate, 4-tert-butyl cyclohexyl acetate, verdox (2-tert-butyl cyclohexyl acetate), vertenex, (4-tert-butylcyclohexyl acetate), Violiff (carbonic acid 4-cycloocten-1-yl methyl ester), ethenyl-iso-amyl carbinylacetate, fenchyl acetate, fenchyl benzoate, fenchyl-n-butyrate, fenchyl isobutyrate, laevo-menthyl acetate, dl-menthyl acetate, menthyl anthranilate, menthyl benzoate, menthyl-iso-butyrate, menthyl formate, laevo-menthyl phenylacetate, menthyl propionate, menthyl salicylate, menthyl-iso-valerate, cyclohexyl acetate, cyclohexyl anthranilate, cyclohexyl benzoate, cyclohexyl butyrate, cyclohexyl-iso-butyrate, cyclohexyl caproate, cyclohexyl cinnamate, cyclohexyl formate, cyclohexyl heptoate, cyclohexyl oxalate, cyclohexyl pelargonate, cyclohexyl phenylacetate, cyclohexyl propionate, cyclohexyl thioglycolate, cyclohexyl valerate, cyclohexyl-iso-valerate, methyl amylacetate, methyl benzyl carbinyl acetate, methyl butyl cyclohexanyl acetate, 5-methyl-3-butyl-tetrahydropyran-4-yl acetate, methyl citrate, methyl-iso-campholate, 2-methyl cyclohexyl acetate, 4-methyl cyclohexyl acetate, 4-methyl cyclohexyl methyl carbinyl acetate, methyl ethyl benzyl carbinyl acetate, 2-methylheptanol-6-acetate, methyl heptenyl acetate, alpha-methyl-n-hexyl carbinyl formate, methyl-2-methylbutyrate, methyl nonyl carbinyl acetate, methyl phenyl carbinyl acetate, methyl phenyl carbinyl anthranilate, methyl phenyl carbinyl benzoate, methyl phenyl carbinyl-n-butyrate, methyl phenyl carbinyl-iso-butyrate, methyl phenyl carbinyl caproate, methyl phenyl carbinyl caprylate, methyl phenyl carbinyl cinnamate, methyl phenyl carbinyl formate, methyl phenyl carbinyl phenylacetate, methyl phenyl carbinyl propionate, methyl phenyl carbinyl salicylate, methyl phenyl carbinyl-iso-valerate, 3-nonanyl acetate, 3-nonenyl acetate, nonane diol-2:3-acetate, nonynol acetate, 2-octanyl acetate, 3-octanyl acetate, n-octyl acetate, secondary-octyl-iso-butyrate, beta-pentenyl acetate, alpha-phenyl allyl acetate, phenylethyl methyl carbinyl-iso-valerate, phenylethyleneglycol diphenylacetate, phenylethyl ethnyl carbinyl acetate, phenylglycol diacetate, seconday-phenylglycol monoacetate, phenylglycol monobenzoate, isopropyl caprate, isopropyl caproate, isporppyl caprylate, isopropyl cinnamate, para-isopropyl cyclohexanyl acetate, propylglycol diacetate, propyleneglycol di-isobutyrate, propyleneglycol dipropionate, isopropyl-n-heptoate, isopropyl-n-hept-1-yne carbonate, isopropyl pelargonate, isopropyl propionate, isopropyl undecylenate, isopropyl-n-valerate, isopropyl-n-valerate, isopropyl-iso-valerate, isopropyl sebacinate, isopulegyl acetate, isopulegyl acetoacetate, isopulegyl isobutyrate, isopulegyl formate, thymyl propionate, alpha-2,4-trimethyl cyclohexane methylacetate, trimethyl cyclohexyl acetate, vanillin triacetate, vanillylidene diacetate, vanillyl vanillate, and mixtures thereof.

“Teriary alcohol” refers to an alcohol molecule wherein the carbon atom carrying the alcoholic hydroxyl group is covalently bonded to three other carbon atoms, namely, having the general structure

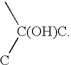

Non-limiting examples of tertiary alcohol ester include tertiary-amyl acetate, caryophyllene acetate, cedrenyl acetate, cedryl acetate, dihydromyrcenyl acetate, dihydroterpinyl acetate, dimethyl benzyl carbinyl acetate, dimethyl benzyl carbinyl isobutyrate, dimethyl heptenyl acetate, dimethyl heptenyl formate, dimethyl heptenyl propionate, dimethyl heptenyl-iso-butyrate, dimethyl phenylethyl carbinyl acetate, dimethyl phenylethyl carbinyl-iso-butyrate, dimethyl phenylethyl carbinyl-iso-valerate, dihydro-nor-dicyclopentadienyl acetate, dimethyl benzul carbinyl butyrate, dimethyl benzyl carbinyl formate, dimethyl benzyl carbinyl propionate, dimethyl phenylethyl carbinyl-n-butyrate, dimethyl phenyletyl carbinyl formate, dimethyl phenylethyl carbinyl propionate, elemyl acetate, ethinyl cyclohexylacetate, eudesmyl acetate, eugenyl cinnamate, eugenyl formate, iso-eugenyl formate, eugenyl phenylacetate, isoeudehyl phenylacetate, guaiyl acetate, hydroxycitronellyl ethylcarbonate, linallyl acetate, linallyl anthranilate, linallyl benzoate, linallyl butyrate, linallyl iosbutyrate, linallyl carproate, linallyl caprylate, linallyl cinnamate, linallyl citronellate, linallyl formate, linallyl heptoate, linallyl-N-methylanthranilate, linallyl methyltiglate, linallyl pelargonate, linallyl phenylacetate, linallyl propionate, linallyl pyruvate, linallyl salicylate, linallyl-n-valerate, linallyl-iso-valerate, methylcyclopentenolone butyrate, methyl cyclopentenolone propionate, methyl ethyl phenyl carbinyl acetate, methyl heptin carbonate, methyl nicotinate, myrcenyl acetate, myrcenyl formate, myrcenyl propionate, cis-ocimenyl acetate, phenyl salicylate, terpinyl acetate, terpinyl anthranilate, terpinyl benzoate, terpinyl-n-butyrate, terpinyl-iso-butyrate, terpinyl cinnamate, terpinyl formate, terpinyl phenylacetate, terpinyl propionate, terpinyl-n-valerate, terpinyl-iso-valerate, tributyl acetylcitrate, and mixtures thereof.

Some alcohols of the unstable alcohol ester perfume ingredients can be both allylic and secondary, or both allylic and tertiary. Non-limiting examples of these ingredients are amyl vinyl carbinyl acetate, amyl vinyl carbinyl propionate, hexyl vinyl carbinyl acetate, 3-nonenyl acetate, 4-hydroxy-2-hexenyl acetate, linallyl anthranilate, linallyl benzoate, linallyl butyrate, linallyl iosbutyrate, linallyl carproate, linallyl caprylate, linallyl cinnamate, linallyl citronellate, linallyl formate, linallyl heptoate, linallyl-N-methylanthranilate, linallyl methyltiglate, linallyl pelargonate, linallyl phenylacetate, linallyl propionate, linallyl pyruvate, linallyl salicylate, linallyl-n-valerate, linallyl-iso-valerate, myrtenyl acetate, nerolidyl acetate, nerolidyl butyrate, beta-pentenyl acetate, alpha-phenyl allyl acetate, and mixtures thereof.

“Allylic ketone” refers to a ketone molecule wherein the ketone functional group is covalently bonded to a carbon-carbon double bond in the alpha and beta positions, namely, having a general structure C—C(═O)—C═C. Non-limiting examples of allylic ketone perfume ingredients include acetyl furan, allethrolone, allyl ionone, allyl pulegone, amyl cyclopentenone, benzylidene acetone, benzylidene acetophenone, alpha iso methyl ionone, 4-(2,6,6-trimetyl-1-cyclohexen-1-yl)-3-buten-2-one, beta damascone (1-(2,6,6-trimethylcyclohexen-1-yl)-2-buten-1-one), damascenone (1-(2,6,6-trimethyl-1,3-cyclohexadien-1-yl)-2-buten-1-one), delta damascone (1-(2,6,6-trimethyl-3-cyclo-hexen-1-yl)-2-buten-1-one), alpha ionone (4-(2,6,6-trimethyl-1-cyclohexenyl-1-yl)-3-buten-2-one), beta ionone (4-(2,6,6-trimethyl-1-cyclohexen-1-yl)-3-butene-2-one), gamma methyl ionone (4-(2,6,6-trimethyl-2-cyclohexyl-1-yl)-3-methyl-3-buten-2-one), pulegone, and mixtures thereof.

“Acetal” refers to an acetal molecule wherein the aldehyde functional group is covalently bonded to two oxygen atoms of two hydroxyl groups at the same carbonyl carbon, namely, having a general structure C—CH(OC)₂. Non-limiting examples of acetal perfume ingredients include acetaldehyde-benzyl-beta-methoxyethyl acetal, acetaldehyde-di-iso-amyl acetal, acetaldehyde-di-pentandeiol acetal, acetaldehyde-di-n-propyl acetal, 10 acetaldehyde-ethyl-trans-3-hexenyl acetal, acetaldehyde-phenylethyleneglycol acetal, acetaldehyde phenylethyl-n-propylacetal, cinnamic aldehyde dimethyl acetal, acetaldehyde-benzyl-beta-methoxyethyl acetal, acetaldehyde-di-iso-amylacetal, acetaldehyde diethylacetal, acetaldehyde-di-cis-3-hexenyl acetal, acetaldehyde-di pentanediol acetal, acetaldehyde-di-n-propyl acetal, acetaldehyde-ethyl-trans-3-hexenyl acetal, acetaldehyde-phenylethyleneglycol acetal, acetaldehyde phenylethyl-n-propylacetal, acetylvanillin dimethylacetal, alpha-amylcinnamic aldehyde-di-iso-propyl acetal, p-tertiary-amyl phenoxy acetaldehyde diethylacetal, anisaldehyde-diethylacetal, anisaldehyde-dimethylacetal, iso-apiole, benzaldehyde diethylacetal, benzaldehyde-di-(ethyleneglycol monobutylether) acetal, benzaldehyde dimethylacetal, benzaldehyde ethyleneglycolacetal, benzaldehyde glyceryl acetal, benzaldehydepropyleneglycol acetal, cinnamic aldehyde diethyl acetal, citral diethyl acetal, citral dimethyl acetal, citral propyleneglycol acetal, alpha-methylcinnamic aldehyde diethylacetal, alpha-cinnamic aldehyde dimethylacetal, phenylacetaldehyde-2,3-butyleneglycol acetal, phenylacetaldehyde citronellyl methyl acetal, phenylacetaldehyde diallylacetal, phenylacetaldehyde diamylacetal, phenylacetaldehyde dibenzylacetal, phenylacetaldehyde dibutyl acetal, phenylacetaldehyde diethylacetal, phenylacetaldehyde digeranylacetal, phenylacetaldehyde dimethylacetal, phenylacetaldehyde ethyleneglycol acetal, phenylacetalde glycerylacetal, citronellal cyclomonoglycolacetal, citronellal diethylacetal, citronellal dimethylacetal, citronellal diphenylethyl acetal, geranoxyacetaldehyde diethylacetal, and mixtures thereof.

“Ketal” refers to a ketal molecule wherein the carbonyl functional group of a ketone is covalently bonded to two oxygen atoms of two hydroxyl groups at the same carbonyl carbon, namely, having a general structure CC(OC)₂C. Non-limiting examples of acetal perfume ingredients include acetone diethylkatal, acetone dimethylketal, acetophenone diethyl ketal, methyl amyl catechol ketal, methyl butyl catechol ketal, and mixtures thereof.

Non-limiting examples of perfume ingredients being condensation products of amines and alhehydes, and not being preferred in the perfume compositions of the present invention include anisaldehyde-methylanthranilate, aurantiol (hydroxycitronellal methylanthranilate), verdantiol (4-tert-butyl-alpha-methyldihydrocinnamaldehyde methyl anthranilate), vertosine (2,4-dimethyl-3-cyclohexene carbaldehyde), hydroxycitronellal ethylanthranilate, hydroxycitronellal linallylanthranilate, methyl-N-(4-(4-hydroxy-4-methylpentyl)-3-cyclohexenyl-methylidene)-anthranilate, methylnaphthylketone-methylanthranilate, methyl nonyl acetaldehyde methylanthranilate, methyl-N-(3,5,5-trimethylhexylidene) anthranilate, vanillin methylanthranilate, and mixtures thereof.

While not wishing to be bound by theory, it is believed that the porous mineral carriers of the present invention exert a catalytic effect that promotes the decomposition of these particular perfume ingredients.

The perfume compositions that are suitable for use in the present invention typically comprises less than about 30%, preferably less than about 15%, more preferably less than about 7%, even more preferably less than about 5%, yet even more preferably less than about 3%, and even more preferably less than about 1%, by weight of the perfume composition, of unstable perfume ingredients, preferably selected from the group consisting of allylic alcohol ester, secondary alcohol ester, tertiary alcohol ester, allylic ketone, condensation product of amines and aldehydes, and mixtures thereof, more preferably, allylic alcohol ester, secondary alcohol ester, tertiary alcohol ester, allylic ketone, acetal, ketal, condensation product of amines and aldehydes, and mixtures thereof.

A “stable” perfume ingredient can be loaded into activated/dehydrated zeolite 13× in the same manner without substantial degradation, with typically at least about 50%, preferably at least 65%, more preferably at least about 80%, and even more preferably at least about 95% of that ingredient not decomposed into other by-products. A perfume molecule is also considered as “stable” when it is isomerized in the zeolite loading process into another structure with the same molecular weight. Non-limiting examples of such stable perfume ingredients include alpha-pinene and beta-pinene.

Thus, the perfume compositions that are suitable for use in the present invention typically comprises at least about 70%, preferably at least about 85%, more preferably at least about 93%, even more preferably at least about 95%, yet even more preferably at least about 97%, and even more preferably at least about 99%, by weight of the perfume composition, of stable perfume ingredients.

Porous mineral carriers provide an advantageous benefit in that they can retain perfume ingredients for a slow release, including non-substantive ingredients. Therefore, preferably, perfume compositions that are incorporated into the porous mineral carrier, for use in the compositions and articles of the present invention comprise at least about 30%, preferably at least about 50%, more preferably at least about 65%, of non-substantive perfume ingredients which are characterized by having a boiling point equal to or lower than about 250° C.

Non-limiting examples of such non-substantive perfume ingredients include amyl acetate, amyl propionate, anethol, anisic aldehyde, anisole, benzaldehyde, benzyl acetate, benzyl acetone, benzyl alcohol, benzyl butyrate, benzyl formate, benzyl iso valerate, benzyl propionate, camphor gum, carvacrol, laevo-carveol, d-carvone, laevo-carvone, citral (neral), citronellol, citronellyl acetate, citronellyl isobutyrate, citronellyl nitrile, citronellyl propionate, para-cresol, para-cresyl methyl ether, cyclohexyl ethyl acetate, cuminic alcohol, cuminic aldehyde, cyclal C (3,5-dimethyl-3-cyclohexene-1-carboxaldehyde), para-cymene, decyl aldehyde, dimethyl benzyl carbinol, dimethyl octanol, diphenyl oxide, dodecalactone, ethyl acetate, ethyl aceto acetate, ethyl amyl ketone, ethyl benzoate, ethyl butyrate, ethyl hexyl ketone, ethyl phenyl acetate, eucalyptol, eugenol, fenchyl alcohol, geraniol, geranyl nitrile, hexenol, beta gamma hexenol, hexenyl acetate, cis-3-hexenyl acetate, hexenyl isobutyrate, cis-3-hexenyl tiglate, hexyl acetate, hexyl formate, hexyl neopentanoate, hexyl tiglate, hydratropic alcohol, hydroxycitronellal, indole, alpha-irone, isoamyl alcohol, isobutyl benzoate, isomenthone, isononyl acetate, isononyl alcohol, isobutyl quinoline, isomenthol, para-isopropyl phenylacetaldehyde, isopulegol, isopulegyl acetate, isoquinoline, cis-jasmone, lauric aldehyde (dodecanal), ligustral (2,4-dimethyl-3-cyclohexene-1-carboxaldehyde), linalool, linalool oxide, menthone, methyl acetophenone, para-methyl acetophenone, methyl amyl ketone, methyl anthranilate, methyl benzoate, methyl benzyl acetate, methyl chavicol, methyl eugenol, methyl heptenone, methyl heptine carbonate, methyl heptyl ketone, methyl hexyl ketone, methyl nonyl acetaldehyde, methyl octyl acetaldehyde, methyl salicylate, myrcene, neral, nerol, gamma-nonalactone, nonyl acetate, nonyl aldehyde, allo-ocimene, octalactone, octyl alcohol (octanol-2), octyl aldehyde, (d-limonene), phenoxy ethanol, phenyl acetaldehyde, phenyl ethyl acetate, phenyl ethyl alcohol, phenyl ethyl dimethyl carbinol, propyl butyrate, rose oxide, 4-terpinenol, alpha-terpineol, terpinolene, tonalid (6-acetyl-1,1,3,4,4,6-hexamethyl tetrahydronaphthalene), undecenal, veratrol (ortho-dimethoxybenzene), and mixtures thereof.

In a preferred embodiment, the perfume composition of the present invention does not consist of 0.2% allyl amyl glycolate, 0.31% damascenone, 0.51% decyl aldehyde, 15.27% dihydro iso jasmonate, 1.02% helional, 14.97% ionone gamma methyl, 20.37% linalool, 1.02% myrcene, 15.27% p.t. bucinal, 0.51% para methyl acetophenone, 20.37% phenyl ethyl alcohol, and 10.18% undecavertol, by weight of the perfume composition. In another preferred embodiment, the perfume composition of the present invention does not consist of 10% benzyl salicylate, 5% coumarin, 2% ethyl vanillin, 10% ethylene brassylate, 15% galaxolide, 20% hexyl cinnamic aldehyde, 10% gamma methyl ionone, 15% lilial, 5% methyl dihydrojasmonate, 5% patchouli, and 3% tonalid.

Perfume compositions useful in the compositions and articles of the present invention preferably comprise less than about 100% aldehyde and/or acetal perfume ingredients. In addition, such perfume compositions preferably comprise less than 45% terpinol, by weight of the perfume composition.

2. Perfume Carrier Particles

It is preferable that at least a major part of the perfume be contained or adsorbed to a porous carrier particle to prevent premature loss, as well as to avoid a strong product perfume odor. The porous carrier particles will comprise between about 1% and about 95%, and preferably between about 20% and about 95% of the air freshening composition. As used herein, “porous carrier particles” or includes porous solids selected from the group consisting of amorphous silicates, crystalline non-layer silicates, layered silicates, calcium carbonates, calcium/sodium carbonate double salts, sodium carbonates, clays, metal oxides (e.g. alumina, aluminates, aluminosilicates), zeolites, sodalites, alkali metal phosphates, macroporous zeolites, chitin microbeads, carboxyalkylcelluloses, carboxyalkylstarches, foams, porous starches, chemically modified starches, starch derivatives, low and high molecular weight sugars, and sugar derivatives, cyclodextrins, fumed and precipitated silicas, and mixtures thereof.

The selection of the most suitable method of perfume delivery takes into account the effectiveness, the efficiency, and the cost of each method. Perfume loaded into zeolite is preferred for its effectiveness, ease of processing, and low cost. The zeolite cavity protects and retains the perfume ingredients from physical effects in the absence of more than about 20% relative humidity (e.g., no rupture/perfume loss during processing, packaging, shipping, and storing of the product, or perfume loss from diffusion) and from chemical effects (e.g., degradation during storage and handling).

Therefore the release of the perfume composition is preferably a moisture activated mechanism wherein the perfume components are released upon the perfume carrier being contacted with liquid or vapor water, preferably water vapor. In addition, it has been discovered that moisture bearing materials referred and described hereinbelow as “moisture providing materials” may be incorporated into the compositions and articles of the present invention to provide moisture that will trigger the perfume release under more dry conditions such as where the relative humidity is less than about 20%. Further still, the release of perfume from the porous carrier particles can be varied by the incorporation of binders and other materials that behave similar to water, having a strong affinity for the pores of the carrier particles, and thus will trigger the release of the perfume composition when present in the composition.

Preferred perfume carrier materials are zeolite X, zeolite Y and mixtures thereof. The term zeolite as used herein refers to a crystalline aluminosilicate material. The structural formula of a zeolite is based on the crystal unit cell, the smallest unit of structure represented by

Mm/n[AlO₂)ₘ(SiO₂)_{y}]×H₂O

where n is the valence of the cation M, x is the number of water molecules per unit cell, m and y are the total number of tetrahedra per unit cell, and y/m is from about 1 to about 100. Most preferably, y/m is from about 1 to about 5. The cation M can be Group IA and Group IIA elements, such as sodium, potassium, magnesium and calcium. The aluminosilicate zeolite materials useful in the practice of this invention are commercially available. The preferred zeolite is a faujasite-type zeolite including Type X Zeolite or Type Y Zeolite, both with nominal pore size of about 8 Angstrom units, typically in the range of from about 7.4 to about 10 Angstrom units. Methods for producing X and Y-type zeolites are well known and available in standard texts.

A preferred crystalline alumino-silicate material is Type X zeolites and can be selected from the following:

(I) Na₈₆[AlO₂]₈₆.(SiO₂)₁₀₆.xH₂O,

(II) K₈₆[AlO₂]₈₆.(SiO₂)₁₀₆.xH₂O,

(III) Ca₄₀Na₆[AlO₂]₈₆.(SiO₂)₁₀₆.xH₂O,

(IV) Sr₂₁Ba₂₂[AlO₂]₈₆.(SiO₂)₁₀₆.xH₂O,

and mixtures thereof, wherein x is from about 0 to about 276. Zeolites of Formula I and II have a nominal pore size or opening of about 8.4 Angstrom units. Zeolites of Formulas III and IV have a nominal pore size or opening of about 8.0 Angstrom units.

Another preferred class of crystalline alumino-silicate materialss are the Type Y zeolites that can be selected from the following:

(V) Na₅₆[AlO₂}₅₆.SiO₂)₁₃₆.xH₂O

(VI) K₅₆[AlO₂]₅₆.(SiO₂)₁₃₆.xH₂O

and mixtures thereof, wherein x is from 0 to about 276. Zeolites of Formulas V and VI have a nominal pore size or opening of 8.0 Angstrom units.

Zeolites useful in the compositions and articles of the present invention have an average particle size from about 0.1 microns to about 250 microns, preferably less than about 100 microns, more preferably less than about 40 microns, and even more preferably having an average particle size of about 5 microns, as measured by standard particle size analysis techniques. Different zeolites have a variety of different sizes and physical characteristics. Mixtures of different zeolites are preferred porous carrier particles for use in the compositions and articles of the present invention given as the different sizes and characteristics will allow greater flexibility in terms of the perfume compositions that may be loaded thereon.

Generally, zeolites useful in the compositions and articles of the present invention are described in U.S. Pat. No. 5,955,419 issued Sept. 21,1999, to Barket, Jr., et al., which is incorporated herein by reference. However, different types of zeolites give different performances as do zeolites that are of a common type but that have different physical properties. The following non-limiting examples of zeolites were evaluated for their perfume retention ability: Zeolite 13×, MSHZ-128, MHSZ-Y and Y-Abscent, all commercially available from UOP; Advera 20 IN, commercially available from PQ Corporation; LSX, commercially available from Zeoline; AX and X, commercially available from Vegobond; CVB 901, CVB 100, CVB 300, CVB 400, CVB 500, CVB 600, CVB 712, CVB 720, CVB 760 and CVB 780, all commercially available from Zeolist; AKZO-1 and AKZO-2, both commercially available from AKZO; and ENG-1, ENG-2, ENG-3, and ENG-4, all commercially available from Engelhard.

While a variety of zeolites having different properties are commercially available, zeolites can also be prepared using methods well known in the art. Specifically, there are three primary methods for synthesis of zeolites, namely, (1) the hydrogel method which employs reactive oxides, soluble silicates, soluble aluminates, and caustic to produce high purity powders or zeolites in a gel matrix; (2) a clay conversion method which employs raw minerals such a kaolin and faujisite, soluble silicates and caustic to produce low to high purity powder or zeolite in clay derived matrix; and (3) processes based on the use of naturally occurring raw materials e.g. natural silica, acid treated clay, volcanic glass, amorphous minerals, to yield high purity powders and zeolites on ceramic supports. A preferred process for making a humidity triggered release zeolite, is the hydrogel method outlined above. A preferred type of zeolite for use in humidity-triggered release of perfume is the X type zeolites, and more preferably, Zeolite 13× available from UOP.

Perfume Entrapment in Zeolite

The zeolites to be used herein preferably contain less than about 10% desorbable water, more preferably less than about 8% desorbable water and even more preferably less than about 5% desorbable water. Such materials may be obtained by first activating/dehydrating by heating the zeolite from about 1500 to about 350° C., optionally at a reduced pressure from about 0.001 to about 20 Torr, for at least about 12 hours. After this “activation”, the perfume active or perfume composition is thoroughly mixed with the activated zeolite and, optionally, heated to about 60° C. for up to two hours to accelerate absorption equilibrium within the zeolite particles. The perfume zeolite mixture is then cooled to room temperature, under controlled humidity conditions, at which time the mixture is in the form of a free flowing powder.

The amount of perfume incorporated into the zeolite carrier is less than about 20%, typically less than about 18.5% and more typically less than about 17% by weight of the perfume entrapped porous carrier particles, given the limits on the pore volume of the zeolite. Excess perfume materials and non-deliverable perfume materials that are not incorporated into zeolite pores are likely to be immediately released to the environment. In the case of perfume articles, an optional excess of “free” perfume will provide a desirable immediate “bloom” of the fragrance upon use of the air freshening article.

The mixing and entrapment of perfume active into the perfume carrier can be carried out using various techniques known in the art of adsorption, absorption, and agglomeration. The perfume active (100% active or diluted in a solvent) can be sprayed onto a bed of powder, followed by mixing. Alternatively, the perfume can be loaded in the vapor or superheated phase. Another option is to use a rotating drum mixer, and spray-on perfume active via single fluid, two-fluid, ultrasonic, or other nozzle technology. One can also use continuous agglomerating equipment, well-known to those familiar in the art, to entrap perfume in the porous carrier particles. Most often, perfume actives are adsorbed or absorbed onto perfume carriers by simply mixing the perfume active with the carrier in a bulk mixer, typically a rotating drum mixer. There are however several limitations when using such equipment for perfume entrapment in zeolites, namely (1) the difficulty of controlling the contact between the perfume active and carrier, (2) the limited ability to control heat transfer, (3) the fracture of perfume carrier particles under intense agitation, (4) inability to operate on a continuous basis, and (5) large space requirement for equipment.

Not to be limited by theory, but it is believed that perfume entrapment in zeolite involves several key physical and chemical transformations including: (1) perfume adsorption onto zeolite surface, (2) perfume diffusion into the zeolite cavities, (3) the “binding” of perfume active to a site in the zeolite cavity, (4) intermolecular interactions which lead to selective entrapment of materials in a specific order, (5) the distortion of aluminosilicate lattice of the zeolite cavity; and (6) the binding of perfume molecules to various sites, near the surface as well as within the diffusion pores and cavity.

Adsorption of perfume molecules into zeolite is governed by two stages, specifically, (1) the energetics during initial entrapment, and (2) entropy management at higher levels of perfume inside the cavity. That is, at low loadings, the perfume molecule that “fits” better into the pore space is able to offer the best energy state, favoring its adsorption. At higher levels of perfume loading, there is increased demand to pack as many molecules as possible in the zeolite cavity and smaller molecules begin to dominate the pore space. This model has been verified by the behavior of several systems, including Xenon/Argon, Xenon/Methane, and Carbon Dioxide/Dichlorofluoromethane.

Moreover, perfume adsorption into the zeolite cavity results in a large exothermic release of energy. Typically, the temperature rise of the bulk powder is about 20° to about 40° C. In turn, the increase in particle temperature influences the selectivity of perfume molecules adsorbed (activation energy requirements for adsorption of specific molecules). By controlling the heat transfer during the perfume entrapment operation it is possible to manipulate (1) the quantity of perfume adsorbed, (2) the selectivity of perfume molecules adsorbed into the cavity, (3) the retention of the adsorbed perfume molecules, and (4) the decomposition of perfume molecules. In order to accomplish the objective of obtaining a higher quantity of perfume components entrapped inside the zeolite cavity, minimize zeolite-catalyzed decomposition of perfume materials, and minimize the loss of more volatile perfume components during entrapment, the zeolite particle should be cooled.

Since the kinetics of adsorption of each perfume active will be different, one needs to first run a “blank” (no heat removal) to prepare a temperature-time profile. From this temperature-time profile, estimate the time at which there is a change in slope (i.e. particle temperature begins to plateau or increase substantially). This is the time at which the particle must be cooled in order to minimize evaporative losses, minimize autocatalytic degradation of perfume materials, and maximize adsorption of perfume components into the zeolite cavity. The amount of heat removed influences the final temperature of the zeolite particle. Since each perfume active will have a different composition of volatile components, the influence of the final temperature on perfume retention and degradation will depend on the perfume composition.

By controlling the heat transfer in this manner, a higher quantity of perfume active can be entrapped in the zeolite cavity. In addition, cooling the zeolite after perfume loading minimizes degradation of perfume components, which may otherwise cause serious quality issues.

By way of example, when perfume active is sprayed onto a bed of zeolite X that is agitated in a drum mixer, the heat transfer mechanism occurs through conduction and free convection. Conductive heat transfer is more effective than free convection. The sheet of powder on which perfume active has been sprayed back-mixes with cooler powder such that there is immediate transfer of heat via conduction. The result is a small temperature rise, typically 110° C. to 20° C. upon targeting a 15% by weight loading of perfume active in zeolite 13X.

In the alternative, a continuous mixer may be used wherein the perfume active and zeolite are contacted in a small volume zone with a very low residence time on the order of 1 to about 10 seconds in that contact zone. In this scenario, the modes of heat transfer again are conduction and convection. However, both modes of heat transfer are inefficient in this plug-flow situation, since all of the powder in the small volume zone will reach a target temperature at generally the same time, such that there is no longer a large temperature gradient to promote conductive heat transfer. The result is a much higher temperature rise for the powder, typically on the order of about 30° C. to about 60° C. This large increase in temperature promotes entrapment of perfume actives that have an activation energy barrier. When heat is removed from the zeolite upon reaching a plateau or autocatalytic temperature, the retention of perfume materials inside the zeolite cavity is greatly improved, and the decomposition of perfume materials is minimized.

The final temperature of the powder appears to be more critical than the medium used for cooling. Media evaluated for cooling are liquid nitrogen, powders that are inert to fragrance adsorption from zeolite X such as sodium citrate, sodium carbonate, citric acid etc., shell-and-tube, and plate heat exchangers. The optimum final temperature of the powder will depend on the specific perfume active used and the effectiveness of liquid/solid contacting in the mixer.

A preferred process for entrapping the perfume active in the perfume carrier employs a continuous mixer that maximizes contact area between the perfume active and the perfume carrier. Ideally, the mean droplet diameter of the atomized perfume active is close to or smaller than the mean particle size of the perfume carrier. In the preferred embodiment of the invention, the size of the perfume carrier is less than about 100 microns, preferably less than about 40, more preferably less than about 30 microns, and even more preferably less than about 5 microns. Also, the heat removal from the perfume carrier commences once the particle has reached a plateau or autocatalytic temperature.

### EXAMPLE 1

### Entrapping Perfume on Porous Carrier Particles—Bench Scale

An amount of about 15 g perfume is slowly added (approx 0.08 g/sec) to about 85 g of zeolite X under high agitation in a conventional kitchen blender. A substantial temperature increase is recorded (at about 20° C.) apparently caused by intensive mixing and heat of adsorption of fragrance materials into the zeolite cage structure. The mixture is allowed to mix until the bulk temperature is about 25° C. A product yield of 100 g is analyzed by Gas Chromatography (after acetone treatment for about 1 hr at about 60° C., and extraction by hexane) with an assay of about 12 wt % total fragrance in zeolite.

### EXAMPLE 2

### Entrapping Perfume on Porous Carrier Particles—Plow Mixer

An amount of about 150 g perfume is added at a rate of about 5 g/sec through a pressure nozzle (about 80 psi, average droplet size of about 90 micrometers) to about 850 g of zeolite X under high agitation in a single batch Loedige plow mixer (about 200 RPM plow, about 2000 RPM chopper, about 300 second cycle time). A cooling jacket at about 20° C. is used to remove the heat generated during perfume entrapment (approx. 280 kJ/kg perfume). An amount of about 1000 g of highly flowable powder is collected and analyzed by Gas Chromatography to yield about 15 wt % total perfume in zeolite. An after simulated wash treatment of zeolite (anionic surfactant wash) and analysis by GC give a result of about 12% perfume inside the zeolite pore cage.

### EXAMPLE 3

### Entrapping Perfume on Porous Carrier Particles—Schugi Mixer

Perfume is added continuously at a rate of about 1.08 g/sec through a two-fluid nozzle (about 30 psi, mean droplet size of about 5 micrometers) simultaneously with about 6.12 g/sec of Zeolite 13× added via a screw feeder. The liquid perfume composition and zeolite powder are contacted in a mixing zone consisting of a shaft with 3 blade components (each component fabricated with 6 blades for intense mixing), and a shaft rotation speed of about 2700-3500 RPM. The residence time of the powder in the “agglomeration” zone is less than about 3 seconds. Substantial heating of the powder is detected during the 3 second residence time, from about 25° to about 80° C. The product is cooled using liquid nitrogen injection to achieve evaporative cooling. Sufficient liquid nitrogen is injected to obtain a product temperature of about 8° C. Gas Chromatography analysis results in about 15.5 wt % total fragrance oil, and about 14.2 wt % fragrance oil inside the zeolite cavity (anionic surfactant wash).

The air freshener articles according to the present invention provide intense, long-lasting fragrance. Conventional hydrophobic porous particles provide controlled release of hydrophobic perfume materials, however, they do not effectively deliver the highly volatile, hydrophilic perfume materials that have a boiling points of less than about 250° C. Zeolite X, on the other hand, can effectively deliver these highly volatile components in a controlled manner as these hydrophilic components have been observed to exhibit the strongest electrostatic interaction with the charge density centers inside the zeolite cavity.

The air freshener articles according to the present invention provide a means to deliver a wider range of perfume characters to the consumer. Since humidity triggers the release of perfume from the zeolite cavity, the formulation of the articles of the present invention can be tailored to provide a gradual release of perfume. In addition, adsorption of water vapor by the zeolite is an exothermic process, causing the zeolite to increase in temperature, and thus provide a natural convection effect to enhance air freshening.

As discussed in detail above, it is believed that the perfume release from the perfume-loaded porous carrier particles is triggered both by humidity and heat. A preferred perfume loaded carrier to achieve this effect is zeolite X. Heating alone (at low ambient moisture, i.e. <about 10% relative humidity) of the perfume loaded zeolite X carrier during the manufacturing of the air freshening article results in nil perfume oil loss, thus providing a controllable mechanism for delivering a variety of highly volatile perfume components. Subsequent exposure of the air freshener composition/article to humidity frees up perfume components for diffusion out of the porous cavity of the carrier particles. Examples below outline preferred embodiments of the article, and characterize the influence of moisture on perfume release.

Air freshening composition of the present invention are ideal for delivering fragrance longevity, wherein the amount of fragrance in the article is from about 0.1 g to about 30 g delivered at a rate of from about 1 mg/hr to about 40 mg/hr per gram of perfume in the air freshening article, released into the surrounding fluid. There are several methods for designing the air freshening article to meet the desired longevity and desired fragrance release rate including: (1) Make sample air freshening article and subject them to expected temperature/humidity conditions for the desired duration, and collect odor intensity and character data over the duration of time, as described in Example 4; and (2) Make several air freshening articles and subject them to the expected temperature/humidity conditions for a short duration of time, remove samples from the temperature/humidity condition at particular times, and characterize the total perfume remaining, and estimate the longevity of the air freshener based on perfume loss rate, as described in Example 5a.

### EXAMPLE 4

### Comparison of Neat Perfume on Carbonate vs. Perfume Entrapped on Porous Carrier Particles

Control:

A sample of about 15 g perfume is sprayed onto about 85 g of sodium carbonate powder (average particle diameter of about 300 micrometers).

Sample A:

A sample of about 15 g perfume is entrapped in about 85 g of Zeolite X using a plow mixer as described in above Example 2.

Sample B:

A sample of about 13 g polyethylene glycol 1450 (Union Carbide) is sprayed onto about 35 g of perfume entrapped in zeolite carrier particles in a Cuisinart Custom 11 kitchen mixer. About 52 g of sodium carbonate (average particle diameter of about 300 micrometers) is then dry mixed with the polyethylene glycol and perfume entrapped zeolite mix to form a powder mixture. About 30 g of the prepared powder mixture is tableted into a cylindrical tablet of diameter of about 45.5 mm and thickness of about 13.6 mm, using a compression force of about 40kN.

An portion of about 10.5 g of the Control, a portion of about 10.5 g of Sample A, and 30 g tablet of Sample B are placed in separate rooms of virtually identical size, temperature, and humidity. Only natural convection of air is allowed in the room, all forced air convection is turned off. The odor intensity and odor character (similarity to the neat perfume oil) in each room is evaluated by an expert perfumer as a function of time. The odor intensities are evaluated according to the following scale and the results are shown in Table A below.

### Odor Intensity Grading Scale

| Intensity | Comment |
|---|---|
| −3.0 | Significantly lower intensity than control. |
| −2.0 | Moderately lower intensity than control. |
| −1.0 | Slightly lower intensity than control. |
| 0 | Equal intensity to control. |
| +1.0 | Slightly higher intensity than control. |
| +2.0 | Moderately higher intensity than control. |
| +3.0 | Significantly higher intensity than control. |

TABLE A

| Time | Control | Sample A | Sample B |
|---|---|---|---|
| 1 hour | Intensity 0 (Assigned) | Intensity +1.5 | Intensity +1.0 |
| | Character - Apple, Green. | Character - Red Apple, Green, | Character - Apple, Green. |
| | Very similar to oil control. | More well rounded character than | Similar to oil control with less |
| | | control. | etherial and acetate notes. |
| | | Similar to oil control with less | |
| | | etherial and acetate notes. | |
| 3 hours | Intensity 0 (Assigned) | Intensity +2.0 | Intensity +2.0 |
| | Character - Very Low | Character - Red Apple, Green, | Character - Apple, Green, |
| | Room Odor, Apple, Green. | More well rounded character than | More Green than 1 hour |
| | Similar to oil control. | control. | evaluation. |
| | | Similar to oil control with less | More similar to oil control with |
| | | etherial and acetate notes. | more green (acetate) notes. |
| 8 hours | Intensity 0 (Assigned) | Intensity +2.5 | Intensity +2.5 |
| | Character - Very Low | Character - Apple, Green, More | Character - Apple, Green, |
| | Room Odor, Apple, Green. | green than 3 hour evaluation. | Slightly more green than |
| | Similar to oil control. | Closer to oil control than 3 hour | Perfume entrapped in zeolite |
| | | evaluation with more etherial and | tablets. |
| | | acetate notes. | Similar to oil control. |
| 24 hours | Intensity 0 (Assigned) | Intensity +2.0 | Intensity +2.0 |
| | Character - Very Low | Character - Apple, Green. | Character - Green, Apple, |
| | Room Odor, Apple, Green. | Similar to oil control with | Slightly more green than |
| | Similar to oil control. | slightly less etherial and acetate | perfume entrapped in zeolite |
| | | notes. | tablets. |
| | | | Similar to oil control with |
| | | | slightly less etherial and |
| | | | acetate notes. |
| 48 hours | Intensity 0 (Assigned) | Intensity +1.5 | Intensity +1.5 |
| | Character - Apple, Green, | Character - Apple, Green. | Character - Apple, Sweet |
| | Slightly more room | Similar to oil control. | Green. Slightly less green than |
| | intensity than 24 hour | | 24 hour evaluation. |
| | evaluation. | | Similar to oil control with |
| | Similar to oil control, | | slightly less etherial notes. |
| 120 hours | Intensity 0 (Assigned) | Intensity +1.5 | Intensity +2.0 |
| | Character - Very Low | Character - Green, Apple. | Character - Green, Apple. |
| | Room Odor, Apple, Green. | Similar to oil control. | Slightly more intensity than |
| | Slightly more green than | | PLZ tablets. |
| | oil control. | | Similar to oil control. |

### EXAMPLE 5

### Analytical Measurement Of Perfume Release Rate

Control:

A sample of about 15 g of perfume is sprayed onto about 85 g of sodium carbonate powder (average particle diameter of about 300 micrometers).

Sample A:

A sample of about 15 g of perfume is loaded into about 85 g of Zeolite X using a plow mixer as described in above example.

Sample B:

A portion of about 13 g of polyethylene glycol 1450 (Union Carbide) is sprayed onto about 35 g of the perfume entrapped in zeolite in a Cuisinart Custom 11 kitchen mixer. About 52 g of sodium carbonate (average particle diameter of about 300 micrometers) is dry mixed with the polyethylene glycol perfume entrapped zeolite mix. A portion of about 10 g of the prepared powder mixture is then tableted into a cylindrical tablet of diameter of about 25.5 mm and thickness of about 9.2 mm, using a compression force of about 40 kN.

Numerous samples (in open petri dishes) of about 5.0 g of Control, about 7.0 g Sample A, and about 10 g of Sample B are placed in petri dishes (diameter of about 3.5 inches, height of about 0.5 inches) and exposed to the nominal 80° F./60% RH constant temperature/constant humidity condition for various times. At specified times (about 1, 3, 8, 24, etc. hours), a sample of the control, Sample A, and Sample B are pulled out of the room. For each the total mass is recorded, quenched with extraction solution, and analytical method outlined below is followed to evaluate the final perfume composition of the hydrated samples. This data is then used to determine the total moisture content and perfume release rate as a function of time:

Mass Balance:

Final Sample Mass−Initial Sample Mass=Moisture Gained−Perfume Lost

Perfume Lost=(Initial Sample Mass)(Initial Perfume Composition)−(Final Sample Mass)(Final Perfume Composition)

Perfume Release Rate=[(Perfume Loss)ₜ₂−(Perfume Loss)ₜ₁]/[t₂-t₁]/Initial Perfume

By way of example, perfume loaded zeolite powder data is summarized below.

TABLE B1
Sample A

| | | Final Perfume | | | |
|---|---|---|---|---|---|
| | | Composition (g | % Perfume | | Perfume |
| Time (hrs) at | Final Sample | perfume/g final | Lost | Moisture | Release |
| 80F/60% RH | Mass (g) | product) | (g) | Content | Rate (mg/g-hr) |
| 0 | 5.0521 | 0.1266 | 0% | 0.00% | 0 |
| 1 | 5.7806 | 0.1115 | 0% | 12.52% | 0 |
| 2 | 6.0090 | 0.101 | 5% | 16.47% | 59 |
| 3 | 6.1459 | 0.0883 | 15% | 19.37% | 100 |
| 7 | 6.1839 | 0.0735 | 29% | 21.30% | 35 |
| 24 | 6.2157 | 0.037 | 64% | 25.31% | 21 |
| 96 | 6.1836 | 0.0201 | 80% | 32.60% | 2.3 |

An identical analysis is performed on Sample B and the results are presented in Table B2.

TABLE B2
Sample B

| | | | | | Perfume Release |
|---|---|---|---|---|---|
| | Final | Final Perfume | | Moisture | Rate (mg perfume |
| Time (hrs) at | Sample | Composition | % Perfume | Content | released/g initial |
| 80F/60% RH | Mass (g) | (wt %) | Lost | (g/g PLZ) | perfume-hr) |
| 0 | 10.023 | 0.04431 | 0 | 0 | 0 |
| 1 | 10.163 | 0.036 | 17% | 0.062 | 176 |
| 8 | 10.3598 | 0.0274 | 36% | 0.14 | 26 |
| 24 | 10.5802 | 0.0215 | 48% | 0.22 | 8 |
| 56 | 10.7311 | 0.0184 | 56% | 0.20 | 2.2 |
| 117 | 10.78969342 | 0.0101 | 75% | 0.31 | 3.1 |

Sample A shows a half-life (about 50% of initial perfume lost) of less than about 24 hours, while sample B has a half-life of greater than about 24 hours. Although initially intense, the perfume release rate for Sample A dies off quickly, while sample B continues to release fragrance controllably beyond 117 hours.

Note that all release rates presented in the tables above are per gram of initial perfume. Hence, increasing the mass of initial perfume will correspondingly increase the perfume release rate. Further, the selection of materials for the air freshening composition can influence the half-life and perfume release rate of the air freshening article, that is the components of the composition can be used to tailor to the desired longevity and perfume release rate.

Although the analysis is not presented here, one can also develop kinetic models to model for perfume release rate as a function of time. The model can then be used to predict the perfume release rate over a long duration of time, and obtain a better estimate of the longevity of the air freshening article.

Although perfume oil can be directly incorporated into the article of the present invention to provide controlled release of fragrance over time, the absence of a trigger mechanism makes it very difficult to control the rate of perfume release from such an article. The article of the present invention uses zeolite as a preferred perfume carrier. Perfume entrapped in zeolite by itself may not be able to deliver a high perfume release rate for long periods of time (4-8 weeks). The perfume loaded zeolite carrier must be put into an appropriate matrix to provide controlled release of the perfume composition. The appropriate matrix is selected such that it can control the rate of moisture penetration into the zeolite cavity and thereby the displacement of the perfume composition.

Not to be limited by theory, the perfume loaded zeolite can be thought of as a well mixed reactor (release kinetics are first order, similar to the output concentration profile from a well mixed reactor, when a quantity of die is instantaneously injected into the inlet stream. Upon adsorption of moisture, these well mixed reactors will free up perfume inside the cavity to diffuse out to the surface of the zeolite. At the surface, the perfume composition is then available to diffuse into the surrounding environment. Given this mechanism (supporting data provided in the next example), controlling the perfume release rate from zeolite is equivalent to controlling the quantity of perfume available on the surface of the zeolite for diffusion into the surrounding environment. In order to control the quantity of perfume on the surface of the zeolite, one needs to manage the moisture adsorption rate by the zeolite. The moisture adsorption rate can be controlled by choice of moisture retarding material, surface area available for adsorption (size, shape, and design of the article), quantity of perfume, chemicals which act to change the porosity of the tablet over time and the presence of any moisture providing materials. Hence, given a duration of time for air freshening, and a desired perfume release rate for that duration of time, one can utilize these “control knobs” to manage moisture in order to develop compositions and articles that achieve the desired criteria.

### EXAMPLE 6

### Perfume Loss Vs. Moisture Content

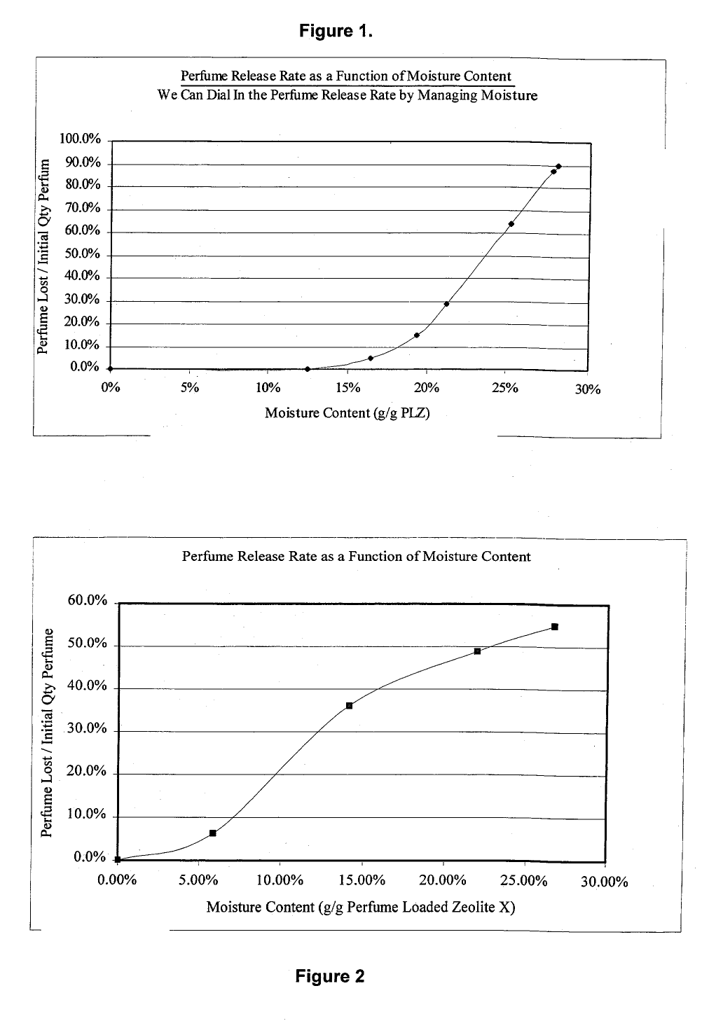

An amount of about 15 g of perfume is loaded into about 85 g of zeolite X in a plow mixer. Several petri dishes containing about 5.0 g powder are exposed to a nominal 80° F./60% RH constant temperature/humidity treatment. The samples are pulled at various times, and analyzed for total perfume remaining. Mass balance is used to determine the moisture content of the powder. The relationship between perfume lost and moisture content is depicted in FIG. 1.

Perfume loaded zeolite does not release perfume until a characteristic moisture content is satisfied (initial moisture content of the zeolite is assumed to be zero in the above analysis). That is, some moisture adsorption is handleable and will not lead to premature release of perfume. This novel learning teaches two key criteria for controlled release

i. By managing moisture adsorption by the zeolite, one can select a desired perfume release rate for the air freshening composition/article; and

ii. It is possible to design-in the initiation of perfume release from the air freshening composition/article.

The above embodiments outline, by way of example, a procedure for determining the product form of the air freshening article given a duration of time for air freshening, and a desired fragrance release rate.

3. Second Component

The compositions and articles of the present invention will contain a second component that may either serve to retard the absorption/adsorption of moisture by the porous carrier particles, serve as a moisture source or provider to the porous carrier particles, and mixtures thereof. This second component will comprise between about 5% and about 80% and preferably between about 30% and about 60% of the air freshening composition. The level of the various second component materials will depend on their identify and function in the compositions and articles as described hereinafter.

a. Moisture Retarding Materials

Moisture retarding materials may take the form of binders that inhibit the adsorption or absorption of moisture by the porous carrier particles, coating materials that retard or inhibit the transmission of moisture to the porous carrier particles and hygroscopic agents or materials that exhibit a strong affinity for moisture and thus serving as a moisture sink to draw moisture away from the porous carrier particles in the compositions and articles. The moisture retarding material can be a binder, or coating material that are used to modify the perfume loaded carrier particles. The moisture retarding material can also be a hygroscopic powder or an inert filler material of controlled particle size.

(1) Binder/Coating Material

The key to controlling the release rate of perfume from the moisture-triggered perfume article is to manage the moisture adsorbed by the porous carrier particles. It should be noted from the following description that there are few distinctions between coatings and binders, either in terms of their composition or properties. A primary compositional difference is that when a material is used as a binder for forming agglomerates, typically less than about 30% of the composition will be comprised of binder material(s). When the material is used as a coating material, a much high level of the material(s) will be present in the composition or article, typically up to about 80% of the composition.

A binder is an agglomerating agent, and may serve several purposes: (1) a chemical added to the perfume carrier particles to increase the overall particle size of the perfume carrier, and thereby reduce the surface area for moisture adsorption, (2) a chemical to which coats perfume carrier particles, thereby acting as a barrier to moisture penetration, (3) a chemical added to perfume carrier particles to aid in making a tablet. Any binder material known in the art can be used. For example highly suitable are materials that have a softening temperature between about 35° C. and about 200° C., but preferably less than about 100° C. and more preferably less than about 65° C. The softening point is defined as the glass transition temperature, if one exists, or the melting temperature.

In general, suitable binders for use herein are those known to those skilled in the art and include anionic surfactants like C₆-C₂₀ alkyl or alkylaryl sulphonates or sulphates, preferably C₈-C₂₀ aklylbenzene sulphonates, fatty acids, cellulose derivatives such as carboxymethylcellulose and homo- or co-polymeric polycarboxylic acid or their salts, nonionic surfactants, preferably C₁-C₂₀ alcohol ethoxylates containing from about 5-100 moles of ethylene oxide per mole of alcohol and more preferably the C₁₅-C₂₀ primary alcohol ethoxylates containing from about 20-100 moles of ethylene oxide per mole of alcohol. Of these tallow alcohol ethoxylated with about 25 moles of ethylene oxide per mole of alcohol (TAE25) or about 50 moles of ethylene oxide per mole of alcohol (TAE50) are preferred. Other preferred binders include the polymeric materials like polyvinylpyrrolidones with an average molecular weight of from about 12,000 to about 700,000 and polyethylene glycols with an average weight of from about 600 to about 10,000. Copolymers of maleic anhydride with ethylene, methylvinyl ether, methacrylic acid or acrylic acid are other examples of polymeric binders. Others binders further include C₁-C₂₀ mono and diglycerol ethers as well as C₁-C₂₀ fatty acids. One can also use water-soluble polymers, such as polyethyleneamines, polyethoxylated amines or imines, which have a softening temperature in the range of from about 35° to about 65° C. More specifically, preferred binder/coating materials can be selected from the group consisting of ethoxylated diamines, glucose, sorbitol, glycerin, polyethylene glycols, polyols, modified starches, and modified starch derivatives, and mixtures thereof. Alternatively, preferred binder/coating materials can be selected from the group consisting of waxes, polyamide resins, aliphatic amides, aliphatic alcohols, divalent alcohols, polyvalent alcohols, emulsifiers, oils, vegetable fats, polypropylene glycol, sugars, fatty acids and combinations thereof.

The porous carrier particles may be processed with barrier technologies such as coating agents or encapsulation to control the release of the perfume active. Non-limiting examples of processes which can be used to encapsulate the porous carrier particles include: spray drying, freeze drying, vacuum drying, extrusion, coacervation, interfacial polymerization, prilling, or other encapsulation processes known in the art. Non-limiting examples of materials suitable for use as a barrier coating include water soluble copolymers such as hydroxylalkyl acrylate or methacrylate, gelatin (U.S. Pat. Nos. 3,681,089 and 3,681,248 and WO 98/28396 A1), polyacrylates, quaternary ammonium salts, acrylic resins, cellulose acetate phthalate, hydrocarbon waxes (U.S. Pat. No. 4,919,441, Marier et al., Apr. 24, 1990, U.S. Pat. No. 5,246,603, Tsaur et al., Sept. 21, 1993, U.S. Pat. No. 5,185,155, Behan et al., Feb. 9, 1993, U.S. Pat. No. 5,500,223, Behan et al., Mar. 19, 1996, EP Nos. 382 464A, 478 326A, 346 034A), urea-formaldehyde resin, polycaprolactone melt, lactic acid, modified starches (U.S. Pat. Nos. 3,971,852, Brenner et al., Jul. 27, 1976 and 5,354,559, Morehouse, Oct. 11, 1994), gums, and hydrolysable polymers. Further, coating agents dislosed in U.S. Pat. No. 6,245,732, Gallon, et al., can also be used to advantage in the compositions and articles of the present invention. The preceding patents and published patent applications and the coating materials described therein are all incorporated herein by reference.

Alternatively, the perfume carrier particles can be uniformly dispersed in a coating material to form a slurry. The slurry can then be prilled. “Prilling” refers to those processes and devices that may be used to atomize liquids and specifically includes dropping the slurry onto a centrifugal atomizer, e.g. spinning disk. The atomized particles are then cooled by chilled air or air at room temperature. Such a process is described in U.S. Pat. No. 5,354,520, which is incorporated herein by reference.

Similarly, the perfume carrier particles can be extruded in a modified starch matrix, as exemplified in U.S. Pat. No. 5,858,959 (Inventor issue date).

Further, especially for an air freshening article in the form of a compressed tablet, preferred binder and coating materials will have rheological properties such that the material will become more fluid during the compression of tablet making. During compression, the temperature of the composition will increase causing the binder to exceed its softening temperature. In this state, preferred binders will be more fluid and will have increased interaction with the other composition components. Further, this change in rheology helps to agglomerate fine particulates with coarser materials to bind them into a tablet form. The characteristics of the composition particles are critical during compression.

The binder or coating material can be chosen based on desired properties of the air freshening article. For example, hydrophilic binders or coating agents tend to draw water into compositions/articles containing them and make water available for adsorption by zeolite in a dense matrix, influencing the perfume release rate. Similarly, hydrophobic binders retard the water adsorption rate of zeolite, and may act as solvents for perfume released by the zeolite, both characteristics that tend to decrease the rate of perfume release from the air freshening article into the surrounding environment. Binder materials that have a low softening temperature may tend to extract perfume materials from within the zeolite cage, or alternatively, displace perfume materials from the cage structure thereby increasing the perfume release rate. Therefore, binders with low softening temperatures may be preferred where the air freshening article is to be used in a low humidity environment (less than about 20-30% RH).

### EXAMPLE 7

### Polyethylene Glycol as Binder

Polyethylene glycol 1500 is a nonionic binder that melts between 45-55° C. Approximately, about 13 g of polyethylene glycol 1500 at about 60° C. is added to about 35 g of perfume loaded zeolite X powder, under high agitation in a Cuisinart Custom 11 kitchen mixer. The agglomerated powder is dry mixed with about 52 g of sodium carbonate and tableted using an Instron (about 60 mm/min compression rate, about 40 kN compaction force, about 25.5 mm diameter cylindrical tablet). Numerous tablets are exposed to nominal 80F/60% RH in open petri dishes. A tablet is pulled from the constant temperature, constant humidity room at various times to measure the total perfume remaining in the tablet. This data, along with mass change data, is used to estimate the perfume release rate as a function of moisture content and is shown in FIG. 2.

The data shows that a binder can be chosen to act similar to water, and to initiate perfume release from the zeolite cavity. Not to be limited by theory, the lower molecular weight chain of polyethylene glycol may diffuse into the zeolite cavity and displace perfume components.

(2) Hygroscopic Powder Materials as Moisture Retardants

For purposes of the present invention “hygroscopic material” refers to a material or combination of materials that undergo a physical and/or chemical change in the presence of water or moisture. A common characteristic of hygroscopic materials is the ability to easily wet, hydrate, or adsorb in the presence of water. Non-limiting examples of such materials are water soluble salts, polymers, starches, dessicants such as amorphous fumed or precipitated silicas, and surfactants.

Other preferred hygroscopic agents or materials include materials that react with one another in the presence of water. The polysaccharides are preferably higher polysaccharides of the non-sweet, colloidally-soluble types, such as natural gums, e.g., gum arabic, starch derivatives, dextrinized and hydrolyzed starches, and the like. The polyhydroxy compounds are preferably alcohols, plant-type sugars, lactones, monoethers, and acetals. Alternatively, desiccants can also be incorporated into the air freshener article composition to control rate of moisture pickup by the porous carrier particle. Nonlimiting examples are: zeolite A, Zeolite X, silica gels, hydrophilic precipitated silicas.

Other materials which are suitable hygroscopic agents include organic and inorganic salts preferably in an anhydrous powder, such as sodium carbonate, sodium sulfate, sodium tripolyphosphate, sodium citrate, sodium bicarbonate, aluminosilicates, layered silicates, alkali metal silicates, calcium carbonate, tetrasodium pyrophosphate, surfactant flakes. Nonlimiting examples of surfactants useful herein include the conventional C11-C₁₈ alkyl benzene sulfonates, and primary, secondary and random alkyl sulfates, the C10-C₁₈ alkyl alkoxy sulfates, the C10-C₁₈ alkyl polyglycosides and their corresponding sulfated polyglycosides, C12-C18 alpha-sulfonated fatty acid esters, C12-C₁₈ alkyl and alkyl phenoxy alkoxylates, C10-C18 amine oxides, and the like. Other conventional useful surfactant flakes are listed in standard texts.

(3) Inert Fillers

Particulate solids can be employed as filler material in the compositions and articles of the present invention. The filler material adds volume to the matrix, but is typically not an active ingredient. The choice of filler material may be made from a wide variety of materials of hardness and particle size range. Notably, the particle size of the powder material can be manipulated to increase or decrease the surface area for water adsorption, which in turn, influences the rate of water adsorption by porous carrier particles. Preferred average particle sizes for inert filler materials are in the range of about 3 to about 1000 microns.

Useful filler materials include minerals such as aluminates and silicates, including alumina, silica, feldspars, clays, carborundum, zircon, clays, quartz and the like and inorganic salts. When used, the solids will preferably comprise about 30-70% by weight of the aqueous reactant phase, most preferably about 40-60%. The weight ratio of solid filler to porous carrier particles is limited only by the ability of the powder mixture of binder, filler and perfume entrapped porous carrier particles to form a robust solid air freshening article.

A preferred filler material for use in the foams of the present invention is NC-4 Feldspar.RTM. (about 170 or 200 mesh) available from Feldspar Corp. of America, Spruce Pine, N.C.

b. Moisture Providing Agents/Materials

Air freshener compositions with moisture retarding materials are ideal for humid environments (>30% RH), as they act to decrease the penetration of moisture into the cavity of the porous carrier, and allow for controlled release of entrapped fragrance. However, for effective air freshening in low humidity environments (e.g. seasonal effects, confined spaces, etc.), incorporation of moisture providing materials enable controlled release of perfume from the moisture-triggered porous carrier. Incorporation of moisture providing materials in the air freshening article is a form of moisture management which utilizes water activity to manipulate the perfume release from the porous carrier. Moisture providing materials insulate or buffer the porous perfume carrier from humidity swings in the ambient fluid, and provides a means to controllably release fragrance.

Moisture providing materials have several key characteristics 1) they have reached their equilibrium hydration when incorporated into the air freshening composition, 2) they act as water “holders”, so that water can be provided to the porous carrier to enable controlled release of fragrance, and 3) they may change form during the process of water exchange with the porous carrier.

The moisture providing material may be selected from partially to fully hydrated forms of organic and inorganic salts including, but not necessarily limited to sodium carbonate monohydrate; aluminum potassium sulfate dodecahydrate (alum); sodium phosphate dodecahydrate; aluminum sulfate octadecahydrate; sodium sulfate decahydrate; sodium carbonate heptahydrate; aluminum ammonium sulfate dodecahydrate; magnesium sulfate docosahydrate; sodium borate decahydrate; sodium metasilicate*9H2O; sodium tetraborate decahydrate; sodium perborate trihydrate; sodium metaborate tetrahydrate; sodium orthophosphate dodecahydrate; sodium dihydrogenphosphate dihydrate; aminocarboxylic acids and their hydrated salts; organic polycarboxylic acids and their hydrated salts, such as, oxalic acid, citric acid, and gluconic acid. In addition, moisture providing materials may also be selected from copolymers of acrylic and maleic and hydrated sodium, potassium, ammonium, or mono or di or triethanolamine salts.

The amount of moisture providing material used in a moisture-triggered air freshening system will depend on a number of factors, but not limited to: (1) the expected water activity of the environment in which the air freshener is to be used, (2) the amount of water in the system that is not bound up in the moisture providing material, (3) the particular moisture providing material used, (4) the particular hygroscopic material and its relative composition in the air freshening system.

While a composition may be conceived where all of the water for perfume release is made available by the moisture providing material, it is expected that in most cases, additional composition elements manage moisture adsorbed by the porous carrier. Typically, the air freshener composition will contain 5% to 80% of the moisture providing material and more preferably 20% to 80% of the moisture providing material by weight of the air freshening system.

4. Optional Materials

The articles of the present invention can also comprise an optional third components which includes optional of adjunct components. The optional components will preferably be selected from the group consisting of free perfumes, pro-fragrances, colorants, insect repelling actives, disintegrants, water swelling agents, porosity modifiers and mixtures thereof, although this group should not be considered as limiting of the optional components that can be used in the compositions and articles of the present invention.

Free Perfumes

Free perfumes or neat perfumes are perfumes that are not incorporated in the porous carrier. Optional free perfume compositions can be useful in the present invention to provide an intense bloom upon exposing the compositions and articles to the atmosphere. Furthermore, many non-diffusive perfume ingredients inherently have a slow rate of release, and do not need to be incorporated, e.g., in a porous carrier. Free perfumes can comprise ingredients that are described in detailed herein above.

Pro-perfumes

The composition and article of the present invention can also optionally and additionally comprise one or more pro-fragrances, pro-perfumes, pro-accords, and mixtures thereof hereinafter known collectively as “pro-perfumes”. The pro-perfumes of the present invention can exhibit varying release rates depending upon the pro-perfume chosen. In addition, the pro-perfumes of the present invention can be admixed with the free perfume materials which are released therefrom to present the user with an initial fragrance, scent, accord, or bouquet.

The pro-perfumes of the present invention can be suitably admixed with any carrier provided the carrier does not catalyze or in other way promote the pre-mature release form the pro-perfume of the perfume raw materials.

The following are non-limiting classes of pro-perfumes according to the present invention.

Esters and polyesters—The esters and polyester pro-perfumes of the present invention are capable of releasing one or more perfume raw material alcohols. Preferred are esters having the formula:

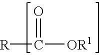

wherein R is substituted or unsubstituted C₁-C₃₀ alkylene, C₂-C₃₀ alkenylene, C₆-C₃₀ arylene, and mixtures thereof; —OR¹ is derived from a perfume raw material alcohol having the formula HOR¹, or alternatively, in the case wherein the index x is greater than 1, R¹ is hydrogen thereby rendering at least one moiety a carboxylic acid, —CO₂H unit, rather than an ester unit; the index x is 1 or greater. Non-limiting examples of preferred polyester pro-perfumes include digeranyl succinate, dicitronellyl succinate, digeranyl adipate, dicitronellyl adipate, and the like.

Beta-Ketoesters—The b-ketoesters of the present invention are capable of releasing one or more perfume raw materials. Preferred b-ketoesters according to the present invention have the formula:

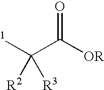

wherein —OR derives from a perfume raw material alcohol; R¹, R², and R³ are each independently hydrogen, C₁-C₃₀ alkyl, C₂-C₃₀ alkenyl, C₁-C₃₀ cycloalkyl, C₂-C₃₀ alkynyl, C₆-C₃₀ aryl, C₇-C₃₀ alkylenearyl, C₃-C₃₀ alkyleneoxyalkyl, and mixtures thereof, provided at least one R¹, R², or R³ is a unit having the formula:

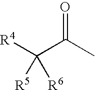

wherein R⁴, R⁵, and R⁶ are each independently hydrogen, C₁-C₃₀ alkyl, C₂-C₃₀ alkenyl, C₁-C₃₀ cycloalkyl, C₁-C₃₀ alkoxy, C₆-C₃₀ aryl, C₇-C₃₀ alkylenearyl, C₃-C₃₀ alkyleneoxyalkyl, and mixtures thereof, or R⁴, R⁵, and R⁶ can be taken together to form a C₃-C₈ aromatic or non-aromatic, heterocyclic or non-heterocyclic ring.

Non-limiting examples of b-ketoesters according to the present invention include 2,6-dimethyl-7-octen-2-yl 3-(4-methoxyphenyl)-3-oxo-propionate; 3,7-dimethyl-1,6-octadien-3-yl 3-(nonanyl)-3-oxo-propionate; 9-decen-1-yl 3-(b-naphthyl)-3-oxo-propionate; (a,a-4-trimethyl-3-cyclohexenyl)methyl 3-(b-naphthyl)-3-oxo-propionate; 3,7-dimethyl-1,6-octadien-3-yl 3-(4-methoxyphenyl)-3-oxo-propionate; 2,6-dimethyl-7-octen-2-yl 3-(b-naphthyl)-3-oxo-propionate; 2,6-dimethyl-7-octen-2-yl 3-(4-nitrophenyl)-3-oxo-propionate; 2,6-dimethyl-7-octen-2-yl 3-(4-methoxyphenyl)-3-oxo-propionate; 3 ,7-dimethyl-1,6-octadien-3-yl 3-(a-naphthyl)-3-oxo-propionate; cis 3-hexen-1-yl 3-(b-naphthyl)-3-oxo-propionate; 2,6-dimethyl-7-octen-2-yl 3-(nonanyl)-3-oxo-propionate; 2,6-dimethyl-7-octen-2-yl 3-oxo-butyrate; 3,7-dimethyl-1,6-octadien-3-yl 3-oxo-butyrate; 2,6-dimethyl-7-octen-2-yl 3-(b-naphthyl)-3-oxo-2-methylpropionate; 3 ,7-dimethyl-1,6-octadien-3-yl 3-(b-naphthyl)-3-oxo-2,2-dimethylpropionate; 3,7-dimethyl-1,6-octadien-3-yl 3-(b-naphthyl)-3-oxo-2-methylpropionate; 3,7-dimethyl-2,6-octadienyl 3-(b-naphthyl)-3-oxo-propionate; 3,7-dimethyl-2,6-octadienyl 3-heptyl-3-oxo-propionate.

Acetals and Ketals—Another class of compound useful as pro-accords according to the present invention are acetals and ketals having the formula:

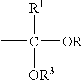

wherein hydrolysis of the acetal or ketal releases one equivalent of aldehyde or ketone and two equivalents of alcohol according to the following scheme:

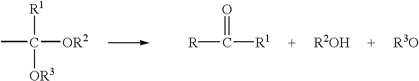

wherein R is C₁-C₂₀ linear alkyl, C₄-C₂₀ branched alkyl, C₆-C₂₀ cyclic alkyl, C₆-C₂₀ branched cyclic alkyl, C₆-C₂₀ linear alkenyl, C₆-C₂₀ branched alkenyl, C₆-C₂₀ cyclic alkenyl, C₆-C₂₀ branched cyclic alkenyl, C₆-C₂₀ substituted or unsubstituted aryl, preferably the moieties which substitute the aryl units are alkyl moieties, and mixtures thereof. R¹ is hydrogen, R, or in the case wherein the pro-accord is a ketal, R and R¹ can be taken together to form a ring. R² and R³ are independently selected from the group consisting of C₅-C₂₀ linear, branched, or substituted alkyl; C₄-C₂₀ linear, branched, or substituted alkenyl; C₅-C₂₀ substituted or unsubstituted cyclic alkyl; C₅-C₂₀ substituted or unsubstituted aryl, C₂-C₄₀ substituted or unsubstituted alkyleneoxy; C₃-C₄₀ substituted or unsubstituted alkyleneoxyalkyl; C₆-C₄₀ substituted or unsubstituted alkylenearyl; C₆-C₃₂ substituted or unsubstituted aryloxy; C₆-C₄₀ substituted or unsubstituted alkyleneoxyaryl; C₆-C₄₀ oxyalkylenearyl; and mixtures thereof.

Non-limiting examples of aldehydes which are releasable by the acetals of the present invention include 4-(4-hydroxy-4-methylpentyl)-3-cyclohexene-1-carboxaldehyde (lyral), phenylacetaldehyde, methylnonyl acetaldehyde, 2-phenylpropan-1-al (hydrotropaldehyde), 3-phenylprop-2-en-1-al (cinnamaldehyde), 3-phenyl-2-pentylprop-2-en-1-al (a-amylcinnamaldehyde), 3-phenyl-2-hexylprop-2-enal (a-hexylcinnamaldehyde), 3-(4-isopropylphenyl)-2-methylpropan-1-al (cyclamen aldehyde), 3-(4-ethylphenyl)-2,2-dimethylpropan-1-al (floralozone), 3-(4-tert-butylphenyl)-2-methylpropanal, 3-(3 ,4-methylenedioxyphenyl)-2-methylpropan-1-al (helional), 3-(4-ethylphenyl)-2,2-dimethylpropanal, 3-(3-isopropylphenyl)butan-1-al (florhydral), 2,6-dimethylhep-5-en-1-al (melonal), n-decanal, n-undecanal, n-dodecanal, 3,7-dimethyl-2,6-octadien-1-al (citral), 4-methoxybenzaldehyde (anisaldehyde), 3-methoxy-4-hydroxybenzaldehyde (vanillin), 3-ethoxy-4-hydroxybenzaldehyde (ethyl vanillin), 3,4-methylenedioxybenzaldehyde (heliotropin), 3,4-dimethoxybenzaldehyde.

Non-limiting examples of ketones which are releasable by the ketals of the present invention include a-damascone, b-damascone, d-damascone, b-damascenone, muscone, 6,7-dihydro-1,1,2,3,3-pentamethyl-4 (5H)-indanone (cashmeran), cis-jasmone, dihydrojasmone, a-ionone, b-ionone, dihydro-b-ionone, g-methyl ionone, a-iso-methyl ionone, 4-(3,4-methylenedioxyphenyl)butan-2-one, 4-(4-hydroxyphenyl)butan-2-one, methyl b-naphthyl ketone, methyl cedryl ketone, 6-acetyl-1,1 ,2,4,4,7-hexamethyltetralin (tonalid), 1-carvone, 5-cyclohexadecen-1-one, acetophenone, decatone, 2-[2-(4-methyl-3-cyclohexenyl-1-yl)propyl]cyclopentan-2-one, 2-sec-butylcyclohexanone, b-dihydro ionone, allyl ionone, a-irone, a-cetone, a-irisone, acetanisole, geranyl acetone, 1-(2-methyl-5-isopropyl-2-cyclohexenyl)-1-propanone, acetyl diisoamylene, methyl cyclocitrone, 4-t-pentyl cyclohexanone, p-t-butylcyclohexanone, o-t-butylcyclohexanone, ethyl amyl ketone, ethyl pentyl ketone, menthone, methyl-7,3-dihydro-2H-1,5-benzodioxepine-3-one, fenchone.

Orthoesters—Another class of compound useful as pro-accords according to the present invention are orthoesters having the formula:

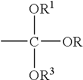

wherein hydrolysis of the orthoester releases one equivalent of an ester and two equivalents of alcohol according to the following scheme:

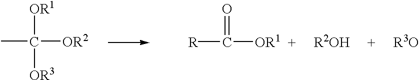

wherein R is hydrogen, C₁-C₂₀ alkyl, C₄-C₂₀ cycloalkyl, C₆-C₂₀ alkenyl, C₆-C₂₀ aryl, and mixtures thereof, R¹, R² and R³ are each independently selected from the group consisting of C₅-C₂₀ linear, branched, or substituted alkyl; C₄-C₂₀ linear, branched, or substituted alkenyl; C₅-C₂₀ substituted or unsubstituted cyclic alkyl; C₅-C₂₀ substituted or unsubstituted aryl, C₂-C₄₀ substituted or unsubstituted alkyleneoxy; C₃-C₄₀ substituted or unsubstituted alkyleneoxyalkyl; C₆-C₄₀ substituted or unsubstituted alkylenearyl; C₆-C₃₂ substituted or unsubstituted aryloxy; C₆-C₄₀ substituted or unsubstituted alkyleneoxyaryl; C₆-C₄₀ oxyalkylenearyl; and mixtures thereof.

Non-limiting examples of orthoester pro-perfumes include tris-geranyl orthoformate, tris(cis-3-hexen-1-yl) orthoformate, tris(phenylethyl) orthoformate, bis(citronellyl) ethyl orthoacetate, tris(citronellyl) orthoformate, tris(cis-6-nonenyl) orthoformate, tris(phenoxyethyl) orthoformate, tris(geranyl, neryl) orthoformate (70:30 geranyl:neryl), tris(9-decenyl) orthoformate, tris(3-methyl-5-phenylpentanyl) orthoformate, tris(6-methylheptan-2-yl) orthoformate, tris([4-(2,2,6-trimethyl-2-cyclohexen-1-yl)-3-buten-2-yl] orthoformate, tris[3-methyl-5-(2,2,3-trimethyl-3-cyclopenten-1-yl)-4-penten-2-yl] orthoformate, trismenthyl orthoformate, tris(4-isopropylcyclohexylethyl-2-yl) orthoformate, tris-(6,8-dimethylnonan-2-yl) orthoformate, tris-phenylethyl orthoacetate, tris(cis-3-hexen-1-yl) orthoacetate, tris(cis-6-nonenyl) orthoacetate, tris-citronellyl orthoacetate, bis(geranyl) benzyl orthoacetate, tris(geranyl) orthoacetate, tris(4-isopropylcyclohexylmethyl) orthoacetate, tris(benzyl) orthoacetate, tris(2,6-dimethyl-5-heptenyl) orthoacetate, bis(cis-3-hexen-1-yl) amyl orthoacetate, and neryl citronellyl ethyl orthobutyrate.

Pro-perfumes are suitably described in the following: U.S. Pat. No. 5,378,468 Suffis et al., issued Jan. 3, 1995; U.S. Pat. No. 5,626,852 Suffis et al., issued May 6, 1997; U.S. Pat. No. 5,710,122 Sivik et al., issued Jan. 20, 1998; U.S. Pat. No. 5,716,918 Sivik et al., issued Feb. 10, 1998; U.S. Pat. No. 5,721,202 Waite et al., issued Feb. 24, 1998; U.S. Pat. No. 5,744,435 Hartman et al., issued Apr. 25, 1998; U.S. Pat. No. 5,756,827 Sivik, issued May 26, 1998; U.S. Pat. No. 5,830,835 Severns et al., issued Nov. 3, 1998; U.S. Pat. No. 5,919,752 Morelli et al., issued Jul. 6, 1999 all of which are incorporated herein by reference.

Conventional colorants and dyes can optionally be incorporated into the compositions and articles of the present invention to provide a desired appearance.

Insect and/or Moth Repelling Agent

The composition of the present invention can optionally contain an effective amount of insect and/or moth repelling agents. Typical insect and moth repelling agents are pheromones, such as anti-aggregation pheromones, and other natural and/or synthetic ingredients. Preferred insect and moth repellent agents useful in the composition of the present invention are perfume ingredients, such as citronellol, citronellal, citral, linalool, cedar extract, geranium oil, sandalwood oil, 2-(diethylphenoxy)ethanol, 1-dodecene, etc. Other examples of insect and/or moth repellents useful in the composition of the present invention are disclosed in U.S. Pat. Nos. 4,449,987, 4,693,890, 4,696,676, 4,933,371, 5,030,660, 5,196,200, and in “Semio Activity of Flavor and Fragrance Molecules on Various Insect Species”, B. D. Mookherjee et al., published in Bioactive Volatile Compounds from Plants, ASC Symposium Series 525, R. Teranishi, R. G. Buttery, and H. Sugisawa, 1993, pp. 35-48, all of said patents and publications being incorporated herein by reference. When an insect and/or moth repellent is used it is typically present at a level of from about 0.005% to about 3%, by weight of the composition. The insect repelling agent can be blended in the free perfume, but is more preferably incorporated in the porous carrier particles.

Disintegrants

The optional disintegrants are useful to provide improved or controlled dissolution. Suitable additional disintegrants include (a) non-cross linked polymeric disintegrants, (b) water-soluble hydrated salts having a solubility in distilled water of at least about 25 g/100 g at about 25° C.; and (c) mixtures thereof.

Preferred non-crosslinked polymeric disintegrants have a particle size distribution such that at least 90% by weight of the disintegrant has a particle size below about 0.3 mm and at least 30% by weight thereof has a particle size below about 0.2 mm. Suitably, the non-crosslinked polymeric disintegrant is selected from starch, cellulose and derivatives thereof, alginates, sugars, polyvinylpyrrolidones, swellable clays and mixtures thereof.

Preferred hydrated salts are selected from hydrates of sodium acetate, sodium metaborate, sodium orthophosphate, sodium dihydrogenphosphate, disodium hydrogen phosphate, sodium potassium tartrate, potassium aluminium sulphate, calcium bromide, calcium nitrate, sodium citrate, potassium citrate and mixtures thereof. Other materials include sodium acetate trihydrate, sodium metaborate tetrahydrate or octahydrate, sodium orthophosphate dodecahydrate, sodium dihydrogen phosphate dihydrate, the di-, hepta- or dodeca-hydrate of disodium hydrogen phosphate, sodium potassium tartrate tetrahydrate, potassium aluminium sulphate dodecahydrate, calcium bromide hexahydrate, tripotassium citrate monohydrate, calcium nitrate tetrahydrate and sodium citrate dihydrate. The hydrated salt can also be selected from water-soluble mono-, di- tri- and tetrahydrate salts and mixtures thereof. Highly preferred herein is sodium acetate trihydrate, tripotassium citrate monohydrate, mixed alkali-metal citrates containing at least one potassium ion and mixtures thereof.

The compositions and articles of the present invention may further comprise one or more optional materials, including but not limited to, (for example hydratable but anhydrous or partially hydrated salts, silica gels), swelling agents (such as disintegrants mentioned above, or spray dried polyvinyl alcohol particles), and mixtures thereof.

Optionally, the air freshener compositions of the present invention can comprise a material that is removed from the composition or articles made therefrom over time, resulting in a change in the density or porosity of the air freshening article. The change in density or porosity of the air freshening article, in turn, can serve as a means for increasing the perfume release rate over long durations of time. Examples of such materials include, but are not limited to fragrance oils, flavor oils, other volatile oils such as volatile silicone oils and mixtures of the same. Alternatively, the porosity changing material can be an acid-base system that reacts in the presence of water to produce a gas which is evolved from the composition or article. An example of one such system is citric acid and sodium carbonate. By way of example, water can be sprayed onto a tablet containing 55:45 citric acid:sodium carbonate co-particle powder system to generate carbon dioxide gas.

Optionally, the air freshener compositions can further comprise a material that swells upon contact with water, and results in an increase in volume of the air freshening article. The increase in volume advantageously increases the surface area available for perfume release, and thus acts to increase the perfume release rate from the air freshening article. Examples of such materials include, but are not limited to natural polymers based on polysaccharide including modified cellulose and cellulose derivatives, starch derivatives, e.g. corn starch, grain starch, potato starch, amylose, amylopectin, dextrin, dextran, hydroxyethyl starch, cationic starch, starch graft polymers and mixtures thereof. In addition, fibrous materials of natural fibers, such as cotton, hemp, wool, and synthetic fibers based on polyvinyl alcohol, polyvinyl acetate, polyvinyl urea, as well as copolymers of these polymers may also be used as water swelling agents or materials. Spray dried polyvinyl alcohol particles (prepared by counter-currently spray drying a slurry of Celvol 205S powder) are also especially useful swelling agents.

Substrates

As described in greater detail below, the compositions and articles of the present invention may further comprise a substrate onto which or into which the porous carrier particles and perfume compositions can be incorporated, as appropriate for their intended use.

Others

As noted above, other optional or adjunct materials can be incorporated into the compositions and articles of the present invention.

B. Processes for Preparing a Solid Air Freshening Article

The present invention is further directed to methods for manufacturing an air freshening article. The methods comprise entrapping a perfume in the porous carrier particles, heating and adding a binder or coating material to form agglomerates, optionally adding a powder inert filler material to form a powder mixture and optionally forming articles from the powder mixture, preferably through compaction. The addition of powder inert filler may be prior to the addition of a binder or coating material. Optionally, the process may further include the step of incorporating a hygroscopic agent into the agglomerates or powder mixture as well as incorporating other optional materials.

A critical aspect of this process is the need to control the humidity conditions under which the perfume is entrapped in the porous carrier particles and during further processing with the perfume entrapped in the porous carrier particles.

The entrapment of the perfume in the porous carrier particles is exemplified in Examples 1-3 above and may simply be accomplished by spraying the perfume compositions on the activated porous carrier particles with agitation. The porous carrier particles with the entrapped perfume may further be encapsulated via various encapsulation processes including spray drying, extrusion, coacervation, interfacial polymerization, suspension polymerizaton, emulsion polymerization, freeze drying, prilling, or other perfume or flavor encapsulation processes. Such encapsulation processes and materials are well described in U.S. Pat. No. 6,025,319 to Surutzidis, et al., U.S. Pat. No. 6,048,830 to Gallon, et al. and U.S. Pat. No. 6,245,732 B1 to Gallon, et al., all commonly assigned to The Procter & Gamble Company each of which is incorporated herein by reference.

In perhaps its most simple form the compositions of the present invention will be free flowing powder having an average particle size of at least about 50 microns. This powder composition may be formed into agglomerates having an average particle size of at least about 150 microns, preferably at least about 300 microns and more preferably at least about 600 microns by incorporating a binder or coating material into the perfume entrapped in porous carrier particles.

Prilling, as described above, is another preferred method for making a coated porous perfume carrier particle wherein the porous carrier particles with entrapped perfume are dispersed in a binder or coating material to form a slurry. The slurry is them poured onto a centrifugal atomizer to form the coated particles which may then be used to form a solid air freshening article.

Further, the compositions of the present invention can be formed into air freshening articles using a variety of extrusion techniques and processes. A conventional extruder wherein the composition is forced through a heated die and broken or cut off in desired lengths as the formed articles emerge from the extruder. Extruder techniques and processes are described in U.S. Pat. No. 5,858,959, which is incorporated herein by reference. Furthermore, the porous carrier particles can be incorporated into foam forming compositions for delivering a controlled release of perfume from the articles formed from the foamed compositons.

Once a powder or coated mixture is obtained, a preferred process for forming a solid article is to compress the mixture into a tablet using conventional tablet making equipment and processes. Alternatively, the mixture may be compacted via press or roller compaction to form a compacted sheet from which articles having a desired shape can be cut using a punch die or other shaped cutter. Solid air freshening articles made from the compositions of the present invention will preferably weigh less than about 200 g, preferably less than about 80 g, and more preferably less than about 40 g. Although moisture may desirably be present in the compositions and articles as bound water within the moisture providing materials, the free water content of the article should be relatively low, preferably less than about 5% by weight of the air freshening article.

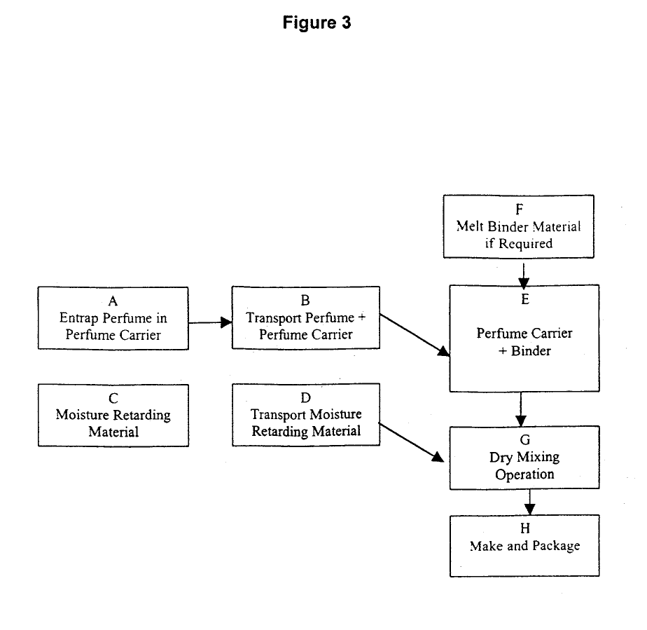

FIG. 3 is a flow chart illustrating the conversion of raw materials into finished compositions and/or articles product. The formulation of a controlled perfume release air freshening article often requires the use of moisture sensitive materials. Controlled humidity conditions may often be required to prevent premature deactivation.

In the preferred embodiment, the performance of processes A, C, E, G, and H require a controlled environment to assure high product quality (sensory performance, minimum loss of perfume oil). A controlled process environment would consist of temperature and humidity control. Preferably, the temperature is maintained below about 80° C., more preferably below about 50° C., and even more preferably less than about 30° C. during these processes. A more critical parameter is the relative humidity in the controlled environment. To ensure minimum deactivation of materials, it is desirable to ensure that the relative humidity is less than about 50%, preferably less than about 40%, and more preferably less than about 30%.

Transport steps B and C require package criteria to minimize deactivation during transport. The package criteria can be determined by following the steps outlined above. The package for transporting raw or converted materials can be a big bag (typically I cubic meter in volume) available in the industry, with a liner. For the preferred embodiment of the invention, the liner meets a specific MVTR criteria, similar to the MVTR required for article stability. Preferably, the liner is glued to the outer bag in such a way that moisture penetration must occur through a continuous layer, and the water vapor transmission rate of the layer is at least less than about 1.2 g H₂O/day/m² and perferably less than about 0.5 g H₂O/day/m²—and more preferably less than about 0.1 g H₂O/day/m², and most preferably less than about 0.02 g H₂O/day/m² to ensure article stability.

Total Perfume Extraction Method

Weigh out 20-30 ml of zeolite, add 200 mls of water, 200 mls of acetone, and 100 mls of internal standard solution. Mix well, add 9.5 ml of acetone and mix again. Place in water bath at 60° C. for 1 hour. Remove from the water bath and allow the solution to cool to room temperature. Attach a 10 ml syringe to a 0.5 micron Millex-LCR filter. Transfer the contents of the scintillation vial into the syringe and filter into a clean scintillation vial. Transfer the filtrate into an autosampler vial and analyze on GC/FID (Gas Chromatography/Flame Ionization Detector).

Determination of Room Odor:

Air freshener articles are evaluated for room odorization using the following method. Air freshening products are placed in cylindrical glass jars with diameter of about 3.5 cm, and height of about 9.5 cm, and placed in a room (width of about 5 ft, length of about 10 ft, height of about 10 ft), with no circulation of air, at an average temperature of 75° F. (±5° F.), and 75% relative humidity (±5% relative humidity). The samples are evaluated for intensity and character relative to the intensity and character of the neat perfume oil.

B. Methods of Use

As noted hereinabove, the compositions and articles of the present invention will provide a controlled release of a perfume composition to an room or environment over a relative long period of time and at a relative uniform rate. Specifically it is envisioned that these compositions can be used to provide a sustained perfume release rate of between about 1 and about 100 mg/hr of perfume, preferably between about 1 and about 50 mg/hr of perfume, per gram of perfume, and that this release rate will be sustained over a period between about 3 days and about 28 days without significant degradation of the perfume intensity or its character.

A specific method of providing a sustained release of a perfume composition will comprise the step of obtaining a composition or article of the present invention and exposing the composition or article to the environment. Atmospheric moisture will contact the porous carrier particles and trigger the release of the perfume composition entrapped therein.

C. Aritcles of Manufacture

The present invention further provides an article of manufacture that comprises a solid air freshening article comprising a composition of the present invention as described hereinbefore, and a humidity resistant package for inhibiting moisture from contacting the porous carrier particles of the air freshening article. The stability of the compositions and articles before their use and their ability to effectively release the perfume components during use requires that such materials be protected from atmospheric moisture with a package having specific moisture barrier characteristics.

Optionally, the compositions and articles of the present invention can further comprise a substrate onto which or into which the porous carrier particles and perfume compositions are incorporated. Such substrates will preferably take the form of a film, foam, sheet, gel, woven or nonwoven fabric, particle or agglomerate, or some combination thereof. Materials and processes for making such substrates are well known in the art relating to air freshening compositions and devices.

Where the perfume is entrapped in a moisture sensitive carrier such as zeolite, the perfume will be desorbed upon adsorption of water, especially water vapor. Water vapor can effectively displace 95-98% of the perfume entrapped inside the zeolite cavity. By way of example, perfume that had been combined with Zeolite 13× (obtained from UOP) was placed in an open petri dish at 80° F. with 70% relative humidity. The amount of perfume initially loaded into the zeolite cavities and the amount of perfume lost due to water absorption are tabulated in Table C below.

TABLE C

| Target Loading of Perfume D in | Perfume Oil Loss after 24 hours at |
|---|---|
| Zeolite X (wt %) | 80F/80% RH (open petri dish) |
| 8% | 97.6% |
| 12% | 96.5% |
| 15% | 95.0% |

The choice of packaging material for a given hygroscopic material and/or perfume carrier can be determined by following several steps. First determine the critical amount of water that can be adsorbed or absorbed by the perfume article without premature loss of perfume, where the loss of perfume can be quantified by an extraction method used to measure the total perfume composition of an article. Water absorption may be determined by exposing the composition/article to constant humidity and determining the mass gained over time. Then, evaluate the performance (analytical and/or sensory) of each perfume article to determine the critical quantity of water. Second, determine the surface area of the package in which the perfume articles will be packaged and sold in the trade. Third, determine the in-trade stability requirement, such as the number of months that the finished product is likely to remain in the package prior to use. The maximum moisture vapor transmission rate (MVTR) for the composition/article may be calculated using the following equation:

MVTR=(Critical Mass of Water)/(Surface Area of Package)/(in-trade stability required) [=] g H₂O/m²/day

Tabulated values of MVTR provided in technical references generally report data determined at about 28-38° C., and about 80%-90% relative humidity such that they represent worse case scenario ambient conditions. Selecting the packaging material under these conditions will ensure long term stability of the article.

Preferably, the article is packaged so that moisture penetration must occur through a continuous layer moisture barrier, and the moisture vapor transmission rate of the layer is at least less than about 1.2 g H₂O/day/m², preferably less than about 0.5 g H₂O/day/m², more preferably less than about 0.1 g H₂O/day/m², and even more preferably less than about 0.02 g H₂O/day/m² to ensure article stability.

The packaging selected to ensure minimal perfume oil loss from the zeolite, must meet several requirements. Films that are permeable to water vapor will not be sufficient to ensure stability. Determination of effective packaging materials must be done on a case-by-case basis since perfume materials will have various odor detection thresholds, and performance benefits that may be detected even after about 20-40% of the oil is lost from the zeolite. To ensure long term stability of about 9 to about 12 months in a sealed package, preferred packaging materials will include bimetallized foil materials, glass, and other materials that are capable of providing the described moisture transfer barrier. Preferred foil materials will have about 7 microns of bimetallized film. It is envisioned that such films will have an opening that is resealable to allow fluid communication between the air freshening article and the environement without having to remove the article from the packaging.

It is envisioned that the article of manufacture may comprise one or more additional air freshening articles of varying weights and dimensions within the same package. Further, it is desirable that the package have a resealable opening or other means for controlling fluid communication between the outside environment and the solid air freshening article.

In a preferred embodiment, the package comprises a container having movable first and second portions each having an opening therein that may be aligned to allow or to control the amount of fluid communication between the air freshening article and the environment. Relative movement between the first and second portions, such as translation or rotation, are preferred for aligning the openings.

In another preferred embodiment, the package comprises a container having an opening and a reclosable lid over the opening wherein the lid may be opened to allow moisture to enter the container and contact the air freshening article. Alternatively or in addition to, the lid can comprise a permeable membrane and a resealable cover for covering the membrane. The membrane made from cloth, wire mesh, and permeable and semi-permeable films that will allow fluid communication therethrough.

In another embodiment of the articles of manufacture will comprise an air freshening articles of the present invention as described hereinbefore, and a package having an opening therein that allows fluid communication between the article and the environment. Such an article is more in the nature of a dispensing device, wherein constant exposure to atmospheric moisture is needed to maintain a desired release rate from the compositions and articles. Thus, the package is preferably a bag made of woven or non-woven fabric(s). Further, the fabric materials are preferably selected from the group consisting of natural and synthetic fibers and mixtures thereof. More specifically, the fabrics may be selected from cloth, nylon, polypropylene, polyethylene, or mixtures thereof. Furthermore, it is envisioned that the bag will be provided with an enlarged opening at one end and will have a draw string or some similar means for closing the opening when desired. Further still, it is envisioned that the bag will comprise a suspending member or means for suspending the bag in a room, closet, automobile or some other enclosed space. A string, rope, wire or loop of similar material may be selected as the suspending member.

In yet another embodiment, the articles of manufacture will comprise an air freshening article and a humidity resistant package, both as described hereinbefore, and a set of instructions associated with the package which comprises an instruction to the user of the article to expose the composition/article to atmospheric moisture to activate the release of the perfume composition to the room. It is envisioned that the set of instructions may further comprise an instruction to apply liquid water to the composition/article to obtain a scent boost from the composition.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An air freshening composition comprising
(a) porous carrier particles having a perfume composition entrapped therein;
(b) a second component for retarding the absorption and/or adsorption of water by the porous carrier particles, said second component selected from the group consisting of:

| | |
|---|---|
| (1) | an inert filler, |
| (2) | a hygroscopic agent; |
| (3) | a binder, |
| (4) | a coating material; |
| (5) | a moisture providing agent; |
| (6) | mixtures thereof; and |

(c) optionally, a third component selected from the group consisting of free perfume, free perfume on various supports, pro-fragrance, colorant, disintegrant, water swelling agent, porosity modifier, insect repelling agent, flavors, and mixtures thereof.

2. The composition of claim 1, wherein the porous carrier particles have an average particle diameter of less than about 100 micrometers.

3. The composition of claim 2, wherein the porous carrier particles have an average particle diameter of less than about 40 micrometers.

4. The composition of claim 1, wherein the porous carrier particles comprise between about 1% and about 95% by weight of the composition.

5. The composition of claim 1, wherein the porous carrier particles are selected from the group consisting of silicas, clays, starches, starch derivatives, sugars, sugar derivatives, zeolites, cyclodextrins, metal oxides (e.g. alumina, aluminates, aluminosilicates), and mixtures thereof.

6. The composition of claim 5, wherein the carrier particles are selected from the group comprising Zeolite X, Zeolite Y, or mixtures thereof.

7. The composition of claim 1, wherein the perfume composition is at least about 1% by weight of the air freshening composition.

8. The composition of claim 1, wherein the perfume composition comprises at least about 25% by weight of the perfume of fragrance material selected from the group consisting of:
a. aromatic and aliphatic esters having molecular weights from about 130 to about 250;
b. aliphatic and aromatic alcohols having molecular weights from about 90 to about 240;
c. aliphatic ketones having molecular weights from about 150 to about 260;
d. aromatic ketones having molecular weights from about 150 to about 270;
e. aromatic and aliphatic lactones having molecular weights from about 130 to about 290;
f. aliphatic aldehydes having molecular weights from about 140 to about 200;
g. aromatic aldehydes having molecular weights from about 90 to about 230;
h. aliphatic and aromatic ethers having molecular weights from about 150 to about 270;
i. condensation products of aldehydes and amines having molecular weights from about 180 to about 320; and
j. mixtures thereof;
wherein said perfume composition is essentially free from nitromusks and halogenated fragrance materials

9. The composition of claim 1, wherein the perfume composition comprises at least about 25% by weight of the perfume composition, of perfume ingredients having boiling point of about 250° C. or lower.

10. The composition of claim 1, wherein the perfume composition comprises less than about 30% by weight of the perfume composition, of unstable perfume ingredients

11. The composition of claim 10, wherein the perfume composition comprises less than about 15% by weight of the perfume composition, of unstable perfume ingredients.

12. The composition of claim 11, wherein the perfume composition comprises less than about 5% by weight of the perfume composition, of unstable perfume ingredients.

13. The composition of claim 10, wherein the unstable perfume ingredients are selected from the group consisting of allylic alcohol ester, secondary alcohol ester, tertiary alcohol ester, allylic ketone, acetal, ketal, condensation product of amines and aldehydes, and mixtures thereof

14. The composition of claim 1, wherein said perfume composition comprises materials that have a dipole-dipole interaction or a nonzero dipole moment.

15. The composition of claim 9, wherein said perfume ingredient is selected from the group consisting of allyl caproate, allyl heptoate, amyl acetate, amyl propionate, anethol, anisic aldehyde, anisole, benzaldehyde, benzyl acetate, benzyl acetone, benzyl alcohol, benzyl butyrate, benzyl formate, benzyl iso valerate, benzyl propionate, camphene, camphor gum, carvacrol, laevo-carveol, d-carvone, laevo-carvone, cinnamyl formate, citral (neral), citronellol, citronellyl acetate, citronellyl isobutyrate, citronellyl nitrile, citronellyl propionate, para-cresol, para-cresyl methyl ether, cyclohexyl ethyl acetate, cuminic alcohol, cuminic aldehyde, 3,5-dimethyl-3-cyclohexene-1-carboxaldehyde, para-cymene, decyl aldehyde, dihydro myrcenol, dihydromyrcenyl acetate, dimethyl benzyl carbinol, dimethyl benzyl carbinyl acetate, dimethyl octanol, diphenyl oxide, dodecalactone, ethyl acetate, ethyl aceto acetate, ethyl amyl ketone, ethyl benzoate, ethyl butyrate, ethyl hexyl ketone, ethyl methyl phenyl glycidate, ethyl phenyl acetate, eucalyptol, eugenol, fenchyl acetate, fenchyl alcohol, tricyclo decenyl acetate, tricyclo decenyl propionate, geraniol, geranyl acetate, geranyl formate, geranyl isobutyrate, geranyl nitrile, hexenol, beta gamma hexenol, hexenyl acetate, cis-3-hexenyl acetate, hexenyl isobutyrate, cis-3-hexenyl tiglate, hexyl acetate, hexyl formate, hexyl neopentanoate, hexyl tiglate, hydratropic alcohol, hydroxycitronellal, indole, alpha-ionone, beta-ionone, gamma-ionone, alpha-irone, isoamyl alcohol, isobomyl acetate, isobutyl benzoate, isomenthone, isononyl acetate, isononyl alcohol, isobutyl quinoline, isomenthol, para-isopropyl phenylacetaldehyde, isopulegol, isopulegyl acetate, isoquinoline, cis-jasmone, lauric aldehyde (dodecanal), 2,4-dimethyl-3-cyclohexene-1-carboxaldehyde, d-limonene, linalool, linalool oxide, linalyl acetate, linalyl formate, menthone, menthyl acetate, methyl acetophenone, para-methyl acetophenone, methyl amyl ketone, methyl anthranilate, methyl benzoate, methyl benzyl acetate, methyl chavicol, methyl eugenol, methyl heptenone, methyl heptine carbonate, methyl heptyl ketone, methyl hexyl ketone, gamma methyl ionone, gamma-n-methyl ionone, alpha-iso gamma-methyl ionone, methyl nonyl acetaldehyde, methyl octyl acetaldehyde, methyl phenyl carbinyl acetate, methyl salicylate, myrcene, neral, nerol, neryl acetate, gamma-nonalactone, nonyl acetate, nonyl aldehyde, allo-ocimene, octalactone, octyl alcohol, octyl aldehyde, orange terpenes, phenoxy ethanol, phenyl acetaldehyde, phenyl ethyl acetate, phenyl ethyl alcohol, phenyl ethyl dimethyl carbinol, alpha-pinene, beta-pinene, prenyl acetate, propyl butyrate, pulegone, rose oxide, safrole, alpha-terpinene, gamma-terpinene, 4-terpinenol, alpha-terpineol, terpinolene, terpinyl acetate, tetrahydro linalool, tetrahydro myrcenol, 6-acetyl-1,1,3,4,4,6-hexamethyl tetrahydronaphthalene, undecenal, ortho-dimethoxybenzene, 2-tert-butylcyclohexyl acetate, 4-tert-butyl cyclohexyl acetate, phenylacetaldehyde dimethylacetal, and mixtures thereof.

16. The composition of claim 9, wherein the perfume composition additionally comprises allyl amyl glycolate, 1,5,5,9-tetramethyl-1,3-oxatricyclotridecane, anethole, 2-ethyl-4-(2,2,3-trimethyl-3-cyclopenten-1-yl)-2-buten-1-ol, benzyl acetone, benzyl salicylate, butyl anthranilate, calone, 2-ethyl-4-(2,2,3-trimethyl-3-cyclopenten-1-yl)-2-buten-1-ol, cinnamic alcohol, coumarin, cyclogalbanate, 3,5-dimethyl-3-cyclohexene-1-carboxaldehyde, 2-methyl-3-(para isopropylphenyl)propionaldehyde, 1-(2,6,6-trimethyl-1,3-cyclohexadien-1-yl)-2-buten-1-one, alpha-damascone, 4-decenal, dihydro isojasmonate, gamma-dodecalactone, ebanol, ethyl anthranilate, ethyl-2-methyl butyrate, ethyl methylphenyl glycidate, ethyl vanillin, eugenol, dihydro-nor-cyclopentadienyl acetate, 3-(3-isopropylphenyl)butanol, ethyl-2-methyl-1,3-dioxolane-2-acetate, dihydro-nor-cyclopentadienyl propionate, heliotropin, 3,3,5-trimethylcyclohexyl-ethyl ether, cis-3-hexenyl salicylate, indole, alpha-ionone, beta-ionone, iso cyclo citral, isoeugenol, alpha-isomethylionone, keone, para-tertiary butyl alpha-methyl hydrocinnamic aldehyde, linalool, 4-(4-hydroxy-4-methyl-pentyl)₃-cylcohexene-1-carboxaldehyde, methyl heptine carbonate, methyl anthranilate, methyl dihydrojasmonate, methyl isobutenyl tetrahydropyran, methyl beta naphthyl ketone, methyl nonyl ketone, beta naphthol methyl ether, nerol, para-anisic aldehyde, para hydroxy phenyl butanone, phenyl acetaldehyde, gamma-undecalactone, undecylenic aldehyde, vanillin, and mixtures thereof.

17. The composition of claim 1, wherein said perfume composition comprises ingredients that can provide an aromatherapy and/or aromachology effect.

18. The composition of claim 1, wherein the perfume composition can alternatively be comprised of a mixture of flavor and perfume components.

19. The composition of claim 1, wherein the second component comprises between about 5% and about 80% by weight of the air freshening composition.

20. The composition of claim 1, wherein the second component comprises a binder, coating agent or mixture thereof having a softening temperature between about 35° C. and about 200° C.

21. The composition of claim 1, wherein the binder is selected from the group consisting of ethoxylated diamines, glucose, sorbitol, glycerin, polyethylene glycols, polyols, modified starches, and modified starch derivatives, waxes, polyamide resins, aliphatic amides, aliphatic alcohols, divalent alcohols, polyvalent alcohols, emulsifiers, oils, vegetable fats, polypropylene glycol, sugars, fatty acids and mixtures.

22. The composition of claim 1, wherein the water-swelling agent comprises a spray dried water-soluble polyvinyl alcohol particles.

23. The composition of claim 1, wherein porosity modifier comprises a volatile oil.

24. The composition of claim 1, wherein the hygroscopic material comprises a amorphous filmed and precipitated silicas, aluminosilicates, aluminates, surfactant flakes, organic and inorganic salts, natural gums, starch derivatives, silica gels, or a mixture thereof.

25. The composition in claim 1, wherein the inert filler material comprises minerals such as aluminates, silicates, inorganic salts.

26. The composition in claim 1, wherein the moisture providing material comprises partially to fully hydrated forms of inorganic and organic salts.

27. The composition of claim 1, wherein the composition is in the form of a free flowing powder having an average particle size of at least about 50 micrometers.

28. The composition of claim 1, wherein the composition is in the form of an agglomerated particle having an average particle size of at least about 150 micrometers.

29. The composition of claim 1, wherein the composition is in the form of a prilled particle

30. The composition of claim 1, wherein the composition is in the form of an extrudate.

31. The composition of claim 1, wherein the composition is in the form of a tablet.

32. The composition of claim 1, wherein the coating material is selected from the group consisting of copolymers, gelatin, polyacrylates, quaternary ammonium salts, acrylic resins, cellulose acetate phthalates, hydrocarbon waxes, urea-formaldehyde resin, polycaprolactone melt, lactic acid, starches, gums, and hydrolysable polymers.

33. The composition of claim 1, wherein the optional pro-perfume is selected from the group consisting of ester pro-perfume, polyester pro-perfume, beta-ketoester pro-perfume, acetal pro-perfume, ketal pro-perfume, orthoester pro-perfume, and mixtures thereof.

34. The composition of claim 1, further comprising a substrate for the porous carrier particles in the form of a film, foam, sheet, gel, woven or nonwoven fabric, particle or agglomerate, and mixtures thereof.

35. The composition of claim 1, wherein the composition is in the form of a solid, flexible foam.

36. A process for preparing a solid air freshening article comprising porous carrier particles having a perfume composition entrapped therein, the process comprising the steps of:
a) entrapping a perfume composition on the porous carrier particles;
b) heating and adding a compatible binder or coating material to the porous carrier particles to form agglomerated particles;
c) optionally adding a powdered filler to the agglomerated particles to form a powder mixture; and
d) optionally forming articles from the powder mixture;
wherein humidity conditions are controlled during steps a) through d), said humidity being maintained below about 50% relative humidity (RH), at a temperature of 25° C.

37. The process of claim 36, wherein entrapment of the perfume in the porous carrier particles comprises spraying on perfume onto the porous carrier particles and/or using a encapsulation process via spray drying, extrusion, coacervation, interfacial polymerization, suspension polymerizaton, emulsion polymerization, freeze drying, prilling, or other perfume or flavor encapsulation processes.

38. The process of claim 36, further comprising the step of incorporating a hygroscopic agent before forming the perfume article.

39. The process of claim 36, wherein the powdered filler is mixed with the perfume entrapped porous carrier particles prior to addition of the binder or coating material.

40. The process of claim 36, wherein the perfume entrapped porous carrier particles are dispersed in the binder or coating material and then prilled to form perfume particles

41. The process of claim 36, further comprising the step of applying free perfume to the powder mixture before forming a perfume article from the prepared powder mixture.

42. The process of claim 36, further comprising the step of incorporating a disintegrant, swelling agent, colorant, or porosity modifier into the powder mixture before forming the perfume article.

43. The process of claim 36, wherein the article is formed by compressing the powder mixture into a tablet.

44. The process of claim 36, wherein the article is formed by compacting the powder mixture into a sheet and cutting the articles from the sheet with a cutter having a desired shape.

45. An article of manufacture for deodorizing or odorizing an environment, the article comprising an air freshening composition according to claim 1 and a humidity resistant package for inhibiting moisture from contacting the air freshening composition.

46. An article of manufacture for deodorizing or odorizing an environment, the article comprising:
A. a solid air freshening article comprising:
porous carrier particles;
a perfume composition adhering to said porous carrier particles;
an optional component selected from the group consisting of fillers, binders, coating materials, hygroscopic agents, moisture providing materials, and mixtures thereof; and
B. a humidity resistant package for inhibiting moisture from contacting the porous carrier particles of the air freshening article.

47. The article of claim 46, wherein the package has a water vapor transmission rate of less than about 1.2 g H₂O/day/m².

48. The article of claim 46, wherein said package is a film.

49. The article of claim 48, wherein the film provides a continuous layer moisture barrier.

50. The article of claim 46, wherein the weight of the solid air freshening article is less than about 200 g.

51. The article of claim 48, further comprising one or more additional air freshening articles of varying weights and dimensions.

52. The article of claim 46, wherein the free water content of the air freshening article is less than about 5% by weight of the article.

53. The article of claim 46, wherein the air freshening article releases perfume at an average rate of about 1 to about 50 mg/hr of perfume, per gram of perfume.

54. The article of claim 46, wherein the humidity resistant package has means to control fluid communication between the outside environment and the solid air freshening article.

55. The article of claim 46, wherein the package comprises first and second portions each having an opening therein that can be aligned to allow fluid communication.

56. The article of claim 55, wherein the first and second portions are rotated relative to each other to align said openings.

57. The article of claim 56, wherein the package comprises a continuous layer moisture barrier with a resealable opening.

58. The article of claim 57, wherein the continuous layer moisture barrier has a water vapor transmission rate of less than about 1.2 g H₂O/day/m².

59. The article of claim 46, wherein the package comprises container with an opening and a reclosable lid over the opening.

60. The article of claim 59, wherein the lid has a membrane to allow fluid communication therethrough and a resealable cover for covering the membrane.

61. The article of claim 60, wherein the membrane is a cloth, wire mesh, semi-permeable membrane or film that allows fluid communication therethrough.

62. The article of claim 46, further comprising a substrate for the porous carrier particles in the form of a film, foam, sheet, gel, woven or nonwoven fabric, particle or agglomerate, and mixtures thereof.

63. An article of manufacture for deodorizing or odorizing an environment, the article comprising
A. a solid air freshening article comprising:
porous carrier particles;
a perfume composition adhering to said porous carrier particles;
an optional component selected from the group consisting of fillers, binders, coating materials, hygroscopic agents, moisture providing materials, and mixtures thereof; and
B. a package for the air freshening article having an opening therein that allows fluid communication between the air freshening article and the environment.

64. The article of claim 63, wherein the package consists of a bag.

65. The article of claim 64, wherein the bag is made of a woven or nonwoven fabric.

66. The article of claim 65, wherein the fabric is made of cloth, nylon, polypropylene, polyethylene or mixtures thereof that will allow for continuous fluid communication.

67. The article of claim 64, wherein the bag has an enlarged opening.

68. The article of claim 66, wherein the bag has means for suspending the bag.

69. An article of manufacture for deodorizing or odorizing an environment, the article comprising
A. a solid air freshening article comprising:
porous carrier particles;
a perfume composition adhering to said porous carrier particles;
an optional component selected from the group consisting of fillers, binders, coating materials, hygroscopic agents, moisture providing materials, and mixtures thereof;
B. a package for the air freshening article; and
C. a set of instructions associated with the package, the set of instructions comprising an instruction to remove the package to expose the composition to humidity and thereby activate the release of the perfume composition to the room.

70. The article of claim 69, wherein the instructions further comprise an instruction to apply liquid water to the composition to obtain a scent boost.
